SEQUENCE LISTING (898 sequences)

SEQ ID NO: 1 - DNA, 444 bp, Homo sapiens
ggagcctaaggaaagagacttgacctggctttcgtcctgcttctgatattcccttctccacaagggctgagagattaggctgcttctccgggatccgcttttccccgggaaacgcgaggatgctccatggagcgtgagcatccaacttttctctcacataaaatctgtctgcccgctctcttggtttttctctgtaaagtaagcaagctgcgtttggcaaataatgaaatggaagtgcaaggaggccaagtcaacaggtggtaacgggttaacaagtgctggcgcggggtccgctagggtggaggctgagaacgccccctcgggtggctggcgcggggttggagacggcccgcgagtgtgagcggcgcctgctcagggtagatagctgagggcgggggtggatgttggatggattagaaccatcacacttgggcctgctgtttg

SEQ ID NO: 2 - DNA, 25 bp, Artificial Sequence
  Forward primer for PCR amplification of bisulfite treated SNCA (-926/-483) DNA
ggagtttaaggaaagagatttgatt

SEQ ID NO: 3 - DNA, 25 bp, Artificial Sequence
  reverse Primer for PCR amplification of SNCA(-926/-483) DNA
caaacaacaaacccaaatataatat

SEQ ID NO: 4 - DNA, 17 bp, Artificial Sequence
  Vector specific primer for sequencing
caggaaacagctatgac

SEQ ID NO: 5 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggggagtttaaggaaa

SEQ ID NO: 6 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggggagtttaaggaaag

SEQ ID NO: 7 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggggagtttaaggaaaga

SEQ ID NO: 8 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggagtttaaggaaagag

SEQ ID NO: 9 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggagtttaaggaaagaga

SEQ ID NO: 10 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagtttaaggaaagagat

SEQ ID NO: 11 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtttaaggaaagagatt

SEQ ID NO: 12 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttaaggaaagagattt

SEQ ID NO: 13 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttaaggaaagagatttg

SEQ ID NO: 14 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttaaggaaagagatttga

SEQ ID NO: 15 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taaggaaagagatttgat

SEQ ID NO: 16 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaggaaagagatttgatt

SEQ ID NO: 17 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggaaagagatttgattt

SEQ ID NO: 18 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggaaagagatttgatttg

SEQ ID NO: 19 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaaagagatttgatttgg

SEQ ID NO: 20 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaagagatttgatttggt

SEQ ID NO: 21 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagagatttgatttggtt

SEQ ID NO: 22 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agagatttgatttggttt

SEQ ID NO: 23 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagatttgatttggtttt

SEQ ID NO: 24 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agatttgatttggtttty

SEQ ID NO: 25 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatttgatttggttttyg

SEQ ID NO: 26 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atttgatttggttttygt

SEQ ID NO: 27 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgatttggttttygtt

SEQ ID NO: 28 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgatttggttttygttt

SEQ ID NO: 29 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgatttggttttygtttt

SEQ ID NO: 30 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatttggttttygttttg

SEQ ID NO: 31 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atttggttttygttttgt

SEQ ID NO: 32 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttggttttygttttgtt

SEQ ID NO: 33 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggttttygttttgttt

SEQ ID NO: 34 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggttttygttttgtttt

SEQ ID NO: 35 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttttygttttgttttt

SEQ ID NO: 36 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttttygttttgtttttg

SEQ ID NO: 37 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttygttttgtttttga

SEQ ID NO: 38 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttygttttgtttttgat

SEQ ID NO: 39 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygttttgtttttgata

SEQ ID NO: 40 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygttttgtttttgatat

SEQ ID NO: 41 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygttttgtttttgatatt

SEQ ID NO: 42 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttttgtttttgatattt

SEQ ID NO: 43 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttgtttttgatatttt

SEQ ID NO: 44 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgtttttgatattttt

SEQ ID NO: 45 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgtttttgatatttttt

SEQ ID NO: 46 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtttttgatattttttt

SEQ ID NO: 47 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttttgatatttttttt

SEQ ID NO: 48 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttgatattttttttt

SEQ ID NO: 49 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttgatatttttttttt

SEQ ID NO: 50 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgatattttttttttt

SEQ ID NO: 51 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgatattttttttttta

SEQ ID NO: 52 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgatatttttttttttat

SEQ ID NO: 53 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatatttttttttttata

SEQ ID NO: 54 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atatttttttttttataa

SEQ ID NO: 55 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tatttttttttttataag

SEQ ID NO: 56 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atttttttttttataagg

SEQ ID NO: 57 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttttttataaggg

SEQ ID NO: 58 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttttttataagggt

SEQ ID NO: 59 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttttataagggtt

SEQ ID NO: 60 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttttataagggttg

SEQ ID NO: 61 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttataagggttga

SEQ ID NO: 62 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttataagggttgag

SEQ ID NO: 63 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttataagggttgaga

SEQ ID NO: 64 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttataagggttgagag

SEQ ID NO: 65 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttataagggttgagaga

SEQ ID NO: 66 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttataagggttgagagat

SEQ ID NO: 67 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tataagggttgagagatt

SEQ ID NO: 68 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ataagggttgagagatta

SEQ ID NO: 69 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taagggttgagagattag

SEQ ID NO: 70 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagggttgagagattagg

SEQ ID NO: 71 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agggttgagagattaggt

SEQ ID NO: 72 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggttgagagattaggtt

SEQ ID NO: 73 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttgagagattaggttg

SEQ ID NO: 74 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttgagagattaggttgt

SEQ ID NO: 75 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgagagattaggttgtt

SEQ ID NO: 76 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgagagattaggttgttt

SEQ ID NO: 77 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagagattaggttgtttt

SEQ ID NO: 78 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agagattaggttgttttt

SEQ ID NO: 79 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagattaggttgtttttt

SEQ ID NO: 80 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agattaggttgtttttty

SEQ ID NO: 81 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gattaggttgttttttyg

SEQ ID NO: 82 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
attaggttgttttttygg

SEQ ID NO: 83 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttaggttgttttttyggg

SEQ ID NO: 84 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taggttgttttttyggga

SEQ ID NO: 85 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggttgttttttygggat

SEQ ID NO: 86 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttgttttttygggatt

SEQ ID NO: 87 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttgttttttygggatty

SEQ ID NO: 88 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgttttttygggattyg

SEQ ID NO: 89 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgttttttygggattygt

SEQ ID NO: 90 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttttttygggattygtt

SEQ ID NO: 91 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttygggattygttt

SEQ ID NO: 92 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttygggattygtttt

SEQ ID NO: 93 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttygggattygttttt

SEQ ID NO: 94 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttygggattygtttttt

SEQ ID NO: 95 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygggattygttttttt

SEQ ID NO: 96 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygggattygtttttttt

SEQ ID NO: 97 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygggattygtttttttty

SEQ ID NO: 98 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggattygttttttttyg

SEQ ID NO: 99 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggattygttttttttygg

SEQ ID NO: 100 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gattygttttttttyggg

SEQ ID NO: 101 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
attygttttttttyggga

SEQ ID NO: 102 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygttttttttygggaa

SEQ ID NO: 103 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygttttttttygggaaa

SEQ ID NO: 104 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygttttttttygggaaay

SEQ ID NO: 105 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttttttttygggaaayg

SEQ ID NO: 106 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttttygggaaaygy

SEQ ID NO: 107 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttygggaaaygyg

SEQ ID NO: 108 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttygggaaaygyga

SEQ ID NO: 109 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttygggaaaygygag

SEQ ID NO: 110 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttygggaaaygygagg

SEQ ID NO: 111 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttygggaaaygygagga

SEQ ID NO: 112 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygggaaaygygaggat

SEQ ID NO: 113 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygggaaaygygaggatg

SEQ ID NO: 114 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygggaaaygygaggatgt

SEQ ID NO: 115 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggaaaygygaggatgtt

SEQ ID NO: 116 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggaaaygygaggatgttt

SEQ ID NO: 117 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaaaygygaggatgtttt

SEQ ID NO: 118 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaaygygaggatgtttta

SEQ ID NO: 119 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaygygaggatgttttat

SEQ ID NO: 120 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aygygaggatgttttatg

SEQ ID NO: 121 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygygaggatgttttatgg

SEQ ID NO: 122 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygaggatgttttatgga

SEQ ID NO: 123 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygaggatgttttatggag

SEQ ID NO: 124 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaggatgttttatggagy

SEQ ID NO: 125 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggatgttttatggagyg

SEQ ID NO: 126 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggatgttttatggagygt

SEQ ID NO: 127 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatgttttatggagygtg

SEQ ID NO: 128 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atgttttatggagygtga

SEQ ID NO: 129 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgttttatggagygtgag

SEQ ID NO: 130 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttttatggagygtgagt

SEQ ID NO: 131 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttatggagygtgagta

SEQ ID NO: 132 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttatggagygtgagtat

SEQ ID NO: 133 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttatggagygtgagtatt

SEQ ID NO: 134 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tatggagygtgagtattt

SEQ ID NO: 135 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atggagygtgagtattta

SEQ ID NO: 136 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggagygtgagtatttaa

SEQ ID NO: 137 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggagygtgagtatttaat

SEQ ID NO: 138 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagygtgagtatttaatt

SEQ ID NO: 139 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agygtgagtatttaattt

SEQ ID NO: 140 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygtgagtatttaatttt

SEQ ID NO: 141 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygtgagtatttaattttt

SEQ ID NO: 142 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtgagtatttaatttttt

SEQ ID NO: 143 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgagtatttaattttttt

SEQ ID NO: 144 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagtatttaatttttttt

SEQ ID NO: 145 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtatttaattttttttt

SEQ ID NO: 146 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtatttaatttttttttt

SEQ ID NO: 147 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tatttaatttttttttta

SEQ ID NO: 148 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atttaattttttttttat

SEQ ID NO: 149 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttaattttttttttata

SEQ ID NO: 150 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttaattttttttttatat

SEQ ID NO: 151 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taattttttttttatata

SEQ ID NO: 152 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aattttttttttatataa

SEQ ID NO: 153 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
attttttttttatataaa

SEQ ID NO: 154 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttttttatataaaa

SEQ ID NO: 155 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttttatataaaat

SEQ ID NO: 156 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttttatataaaatt

SEQ ID NO: 157 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttatataaaattt

SEQ ID NO: 158 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttatataaaatttg

SEQ ID NO: 159 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttatataaaatttgt

SEQ ID NO: 160 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttatataaaatttgtt

SEQ ID NO: 161 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttatataaaatttgttt

SEQ ID NO: 162 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttatataaaatttgtttg

SEQ ID NO: 163 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tatataaaatttgtttgt

SEQ ID NO: 164 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atataaaatttgtttgtt

SEQ ID NO: 165 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tataaaatttgtttgtty

SEQ ID NO: 166 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ataaaatttgtttgttyg

SEQ ID NO: 167 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taaaatttgtttgttygt

SEQ ID NO: 168 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaaatttgtttgttygtt

SEQ ID NO: 169 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaatttgtttgttygttt

SEQ ID NO: 170 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aatttgtttgttygtttt

SEQ ID NO: 171 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atttgtttgttygttttt

SEQ ID NO: 172 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgtttgttygtttttt

SEQ ID NO: 173 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgtttgttygttttttt

SEQ ID NO: 174 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtttgttygtttttttg

SEQ ID NO: 175 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttgttygtttttttgg

SEQ ID NO: 176 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgttygtttttttggt

SEQ ID NO: 177 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgttygtttttttggtt

SEQ ID NO: 178 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgttygtttttttggttt

SEQ ID NO: 179 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttygtttttttggtttt

SEQ ID NO: 180 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygtttttttggttttt

SEQ ID NO: 181 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygtttttttggtttttt

SEQ ID NO: 182 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygtttttttggttttttt

SEQ ID NO: 183 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttttttggtttttttt

SEQ ID NO: 184 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttggttttttttt

SEQ ID NO: 185 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttggtttttttttg

SEQ ID NO: 186 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttggtttttttttgt

SEQ ID NO: 187 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttggtttttttttgta

SEQ ID NO: 188 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttggtttttttttgtaa

SEQ ID NO: 189 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggtttttttttgtaaa

SEQ ID NO: 190 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggtttttttttgtaaag

SEQ ID NO: 191 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtttttttttgtaaagt

SEQ ID NO: 192 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttttttttgtaaagta

SEQ ID NO: 193 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttttgtaaagtaa

SEQ ID NO: 194 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttttgtaaagtaag

SEQ ID NO: 195 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttttgtaaagtaagt

SEQ ID NO: 196 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttgtaaagtaagta

SEQ ID NO: 197 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttgtaaagtaagtaa

SEQ ID NO: 198 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttgtaaagtaagtaag

SEQ ID NO: 199 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgtaaagtaagtaagt

SEQ ID NO: 200 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgtaaagtaagtaagtt

SEQ ID NO: 201 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtaaagtaagtaagttg

SEQ ID NO: 202 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtaaagtaagtaagttgy

SEQ ID NO: 203 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taaagtaagtaagttgyg

SEQ ID NO: 204 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaagtaagtaagttgygt

SEQ ID NO: 205 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagtaagtaagttgygtt

SEQ ID NO: 206 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtaagtaagttgygttt

SEQ ID NO: 207 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtaagtaagttgygtttg

SEQ ID NO: 208 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taagtaagttgygtttgg

SEQ ID NO: 209 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagtaagttgygtttggt

SEQ ID NO: 210 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtaagttgygtttggta

SEQ ID NO: 211 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtaagttgygtttggtaa

SEQ ID NO: 212 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taagttgygtttggtaaa

SEQ ID NO: 213 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagttgygtttggtaaat

SEQ ID NO: 214 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agttgygtttggtaaata

SEQ ID NO: 215 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttgygtttggtaaataa

SEQ ID NO: 216 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgygtttggtaaataat

SEQ ID NO: 217 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgygtttggtaaataatg

SEQ ID NO: 218 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygtttggtaaataatga

SEQ ID NO: 219 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygtttggtaaataatgaa

SEQ ID NO: 220 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttggtaaataatgaaa

SEQ ID NO: 221 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttggtaaataatgaaat

SEQ ID NO: 222 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggtaaataatgaaatg

SEQ ID NO: 223 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggtaaataatgaaatgg

SEQ ID NO: 224 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtaaataatgaaatgga

SEQ ID NO: 225 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtaaataatgaaatggaa

SEQ ID NO: 226 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taaataatgaaatggaag

SEQ ID NO: 227 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaataatgaaatggaagt

SEQ ID NO: 228 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aataatgaaatggaagtg

SEQ ID NO: 229 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ataatgaaatggaagtgt

SEQ ID NO: 230 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taatgaaatggaagtgta

SEQ ID NO: 231 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aatgaaatggaagtgtaa

SEQ ID NO: 232 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atgaaatggaagtgtaag

SEQ ID NO: 233 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgaaatggaagtgtaagg

SEQ ID NO: 234 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaaatggaagtgtaagga

SEQ ID NO: 235 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaatggaagtgtaaggag

SEQ ID NO: 236 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aatggaagtgtaaggagg

SEQ ID NO: 237 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atggaagtgtaaggaggt

SEQ ID NO: 238 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggaagtgtaaggaggtt

SEQ ID NO: 239 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggaagtgtaaggaggtta

SEQ ID NO: 240 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaagtgtaaggaggttaa

SEQ ID NO: 241 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagtgtaaggaggttaag

SEQ ID NO: 242 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtgtaaggaggttaagt

SEQ ID NO: 243 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtgtaaggaggttaagtt

SEQ ID NO: 244 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtaaggaggttaagtta

SEQ ID NO: 245 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtaaggaggttaagttaa

SEQ ID NO: 246 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taaggaggttaagttaat

SEQ ID NO: 247 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaggaggttaagttaata

SEQ ID NO: 248 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggaggttaagttaatag

SEQ ID NO: 249 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggaggttaagttaatagg

SEQ ID NO: 250 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaggttaagttaataggt

SEQ ID NO: 251 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggttaagttaataggtg

SEQ ID NO: 252 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttaagttaataggtgg

SEQ ID NO: 253 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttaagttaataggtggt

SEQ ID NO: 254 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttaagttaataggtggta

SEQ ID NO: 255 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taagttaataggtggtaa

SEQ ID NO: 256 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagttaataggtggtaay

SEQ ID NO: 257 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agttaataggtggtaayg

SEQ ID NO: 258 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttaataggtggtaaygg

SEQ ID NO: 259 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttaataggtggtaayggg

SEQ ID NO: 260 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taataggtggtaaygggt

SEQ ID NO: 261 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aataggtggtaaygggtt

SEQ ID NO: 262 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ataggtggtaaygggtta

SEQ ID NO: 263 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taggtggtaaygggttaa

SEQ ID NO: 264 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggtggtaaygggttaat

SEQ ID NO: 265 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtggtaaygggttaata

SEQ ID NO: 266 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtggtaaygggttaataa

SEQ ID NO: 267 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggtaaygggttaataag

SEQ ID NO: 268 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtaaygggttaataagt

SEQ ID NO: 269 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtaaygggttaataagtg

SEQ ID NO: 270 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taaygggttaataagtgt

SEQ ID NO: 271 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaygggttaataagtgtt

SEQ ID NO: 272 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aygggttaataagtgttg

SEQ ID NO: 273 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygggttaataagtgttgg

SEQ ID NO: 274 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggttaataagtgttggy

SEQ ID NO: 275 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttaataagtgttggyg

SEQ ID NO: 276 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttaataagtgttggygy

SEQ ID NO: 277 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttaataagtgttggygyg

SEQ ID NO: 278 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taataagtgttggygygg

SEQ ID NO: 279 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aataagtgttggygyggg

SEQ ID NO: 280 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ataagtgttggygygggg

SEQ ID NO: 281 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
taagtgttggygyggggt

SEQ ID NO: 282 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aagtgttggygyggggtt

SEQ ID NO: 283 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtgttggygyggggtty

SEQ ID NO: 284 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtgttggygyggggttyg

SEQ ID NO: 285 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgttggygyggggttygt

SEQ ID NO: 286 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttggygyggggttygtt

SEQ ID NO: 287 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggygyggggttygtta

SEQ ID NO: 288 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggygyggggttygttag

SEQ ID NO: 289 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggygyggggttygttagg

SEQ ID NO: 290 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygyggggttygttaggg

SEQ ID NO: 291 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygyggggttygttagggt

SEQ ID NO: 292 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gyggggttygttagggtg

SEQ ID NO: 293 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
yggggttygttagggtgg

SEQ ID NO: 294 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggggttygttagggtgga

SEQ ID NO: 295 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggttygttagggtggag

SEQ ID NO: 296 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttygttagggtggagg

SEQ ID NO: 297 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttygttagggtggaggt

SEQ ID NO: 298 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygttagggtggaggtt

SEQ ID NO: 299 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygttagggtggaggttg

SEQ ID NO: 300 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygttagggtggaggttg

SEQ ID NO: 301 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygttagggtggaggttga

SEQ ID NO: 302 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttagggtggaggttgag

SEQ ID NO: 303 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttagggtggaggttgaga

SEQ ID NO: 304 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tagggtggaggttgagaa

SEQ ID NO: 305 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agggtggaggttgagaay

SEQ ID NO: 306 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggtggaggttgagaayg

SEQ ID NO: 307 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtggaggttgagaaygt

SEQ ID NO: 308 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtggaggttgagaaygtt

SEQ ID NO: 309 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggaggttgagaaygttt

SEQ ID NO: 310 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggaggttgagaaygtttt

SEQ ID NO: 311 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaggttgagaaygttttt

SEQ ID NO: 312 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aggttgagaaygtttttt

SEQ ID NO: 313 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttgagaaygtttttty

SEQ ID NO: 314 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttgagaaygttttttyg

SEQ ID NO: 315 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgagaaygttttttygg

SEQ ID NO: 316 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgagaaygttttttyggg

SEQ ID NO: 317 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagaaygttttttygggt

SEQ ID NO: 318 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agaaygttttttygggtg

SEQ ID NO: 319 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaaygttttttygggtgg

SEQ ID NO: 320 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aaygttttttygggtggt

SEQ ID NO: 321 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aygttttttygggtggtt

SEQ ID NO: 322 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygttttttygggtggttg

SEQ ID NO: 323 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttttttygggtggttgg

SEQ ID NO: 324 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttttygggtggttggy

SEQ ID NO: 325 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttttygggtggttggyg

SEQ ID NO: 326 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttttygggtggttggygy

SEQ ID NO: 327 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttygggtggttggygyg

SEQ ID NO: 328 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygggtggttggygygg

SEQ ID NO: 329 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygggtggttggygyggg

SEQ ID NO: 330 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygggtggttggygygggg

SEQ ID NO: 331 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggtggttggygyggggt

SEQ ID NO: 332 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtggttggygyggggtt

SEQ ID NO: 333 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtggttggygyggggttg

SEQ ID NO: 334 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggttggygyggggttgg

SEQ ID NO: 335 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttggygyggggttgga

SEQ ID NO: 336 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttggygyggggttggag

SEQ ID NO: 337 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggygyggggttggaga

SEQ ID NO: 338 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggygyggggttggagay

SEQ ID NO: 339 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggygyggggttggagayg

SEQ ID NO: 340 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygyggggttggagaygg

SEQ ID NO: 341 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygyggggttggagayggt

SEQ ID NO: 342 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gyggggttggagayggtt

SEQ ID NO: 343 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
yggggttggagayggtty

SEQ ID NO: 344 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggggttggagayggttyg

SEQ ID NO: 345 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggttggagayggttygy

SEQ ID NO: 346 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttggagayggttygyg

SEQ ID NO: 347 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttggagayggttygyga

SEQ ID NO: 348 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggagayggttygygag

SEQ ID NO: 349 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggagayggttygygagt

SEQ ID NO: 350 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggagayggttygygagtg

SEQ ID NO: 351 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagayggttygygagtgt

SEQ ID NO: 352 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agayggttygygagtgtg

SEQ ID NO: 353 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gayggttygygagtgtga

SEQ ID NO: 354 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ayggttygygagtgtgag

SEQ ID NO: 355 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
yggttygygagtgtgagy

SEQ ID NO: 356 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggttygygagtgtgagyg

SEQ ID NO: 357 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttygygagtgtgagygg

SEQ ID NO: 358 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttygygagtgtgagyggy

SEQ ID NO: 359 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tygygagtgtgagyggyg

SEQ ID NO: 360 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygygagtgtgagyggygt

SEQ ID NO: 361 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygagtgtgagyggygtt

SEQ ID NO: 362 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygagtgtgagyggygttt

SEQ ID NO: 363 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagtgtgagyggygtttg

SEQ ID NO: 364 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agtgtgagyggygtttgt

SEQ ID NO: 365 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtgtgagyggygtttgtt

SEQ ID NO: 366 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtgagyggygtttgttt

SEQ ID NO: 367 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtgagyggygtttgttta

SEQ ID NO: 368 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgagyggygtttgtttag

SEQ ID NO: 369 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagyggygtttgtttagg

SEQ ID NO: 370 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agyggygtttgtttaggg

SEQ ID NO: 371 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gyggygtttgtttagggt

SEQ ID NO: 372 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
yggygtttgtttagggta

SEQ ID NO: 373 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggygtttgtttagggtag

SEQ ID NO: 374 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygtttgtttagggtaga

SEQ ID NO: 375 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygtttgtttagggtagat

SEQ ID NO: 376 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttgtttagggtagata

SEQ ID NO: 377 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgtttagggtagatag

SEQ ID NO: 378 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgtttagggtagatagt

SEQ ID NO: 379 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtttagggtagatagtt

SEQ ID NO: 380 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttagggtagatagttg

SEQ ID NO: 381 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttagggtagatagttga

SEQ ID NO: 382 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttagggtagatagttgag

SEQ ID NO: 383 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tagggtagatagttgagg

SEQ ID NO: 384 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agggtagatagttgaggg

SEQ ID NO: 385 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggtagatagttgagggy

SEQ ID NO: 386 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtagatagttgagggyg

SEQ ID NO: 387 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtagatagttgagggygg

SEQ ID NO: 388 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tagatagttgagggyggg

SEQ ID NO: 389 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agatagttgagggygggg

SEQ ID NO: 390 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatagttgagggyggggg

SEQ ID NO: 391 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atagttgagggygggggt

SEQ ID NO: 392 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tagttgagggygggggtg

SEQ ID NO: 393 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agttgagggygggggtgg

SEQ ID NO: 394 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttgagggygggggtgga

SEQ ID NO: 395 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgagggygggggtggat

SEQ ID NO: 396 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgagggygggggtggatg

SEQ ID NO: 397 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gagggygggggtggatgt

SEQ ID NO: 398 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agggygggggtggatgtt

SEQ ID NO: 399 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggygggggtggatgttg

SEQ ID NO: 400 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggygggggtggatgttgg

SEQ ID NO: 401 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gygggggtggatgttgga

SEQ ID NO: 402 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ygggggtggatgttggat

SEQ ID NO: 403 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggggtggatgttggatg

SEQ ID NO: 404 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggggtggatgttggatgg

SEQ ID NO: 405 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggtggatgttggatgga

SEQ ID NO: 406 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtggatgttggatggat

SEQ ID NO: 407 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtggatgttggatggatt

SEQ ID NO: 408 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggatgttggatggatta

SEQ ID NO: 409 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggatgttggatggattag

SEQ ID NO: 410 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatgttggatggattaga

SEQ ID NO: 411 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atgttggatggattagaa

SEQ ID NO: 412 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgttggatggattagaat

SEQ ID NO: 413 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttggatggattagaatt

SEQ ID NO: 414 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttggatggattagaatta

SEQ ID NO: 415 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggatggattagaattat

SEQ ID NO: 416 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggatggattagaattatt

SEQ ID NO: 417 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gatggattagaattatta

SEQ ID NO: 418 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atggattagaattattat

SEQ ID NO: 419 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tggattagaattattata

SEQ ID NO: 420 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggattagaattattatat

SEQ ID NO: 421 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gattagaattattatatt

SEQ ID NO: 422 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
attagaattattatattt

SEQ ID NO: 423 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttagaattattatatttg

SEQ ID NO: 424 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tagaattattatatttgg

SEQ ID NO: 425 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
agaattattatatttggg

SEQ ID NO: 426 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gaattattatatttgggt

SEQ ID NO: 427 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
aattattatatttgggtt

SEQ ID NO: 428 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
attattatatttgggttt

SEQ ID NO: 429 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttattatatttgggtttg

SEQ ID NO: 430 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tattatatttgggtttgt

SEQ ID NO: 431 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
attatatttgggtttgtt

SEQ ID NO: 432 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttatatttgggtttgttg

SEQ ID NO: 433 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tatatttgggtttgttgt

SEQ ID NO: 434 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atatttgggtttgttgtt

SEQ ID NO: 435 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tatttgggtttgttgttt

SEQ ID NO: 436 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
atttgggtttgttgtttg

SEQ ID NO: 437 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgggtttgttgtttgt

SEQ ID NO: 438 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgggtttgttgtttgtt

SEQ ID NO: 439 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgggtttgttgtttgttt

SEQ ID NO: 440 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gggtttgttgtttgtttg

SEQ ID NO: 441 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ggtttgttgtttgtttga

SEQ ID NO: 442 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttgttgtttgtttgag

SEQ ID NO: 443 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgttgtttgtttgagt

SEQ ID NO: 444 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgttgtttgtttgagtt

SEQ ID NO: 445 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgttgtttgtttgagttt

SEQ ID NO: 446 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gttgtttgtttgagtttg

SEQ ID NO: 447 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgtttgtttgagtttga

SEQ ID NO: 448 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tgtttgtttgagtttgaa

SEQ ID NO: 449 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
gtttgtttgagtttgaat

SEQ ID NO: 450 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
tttgtttgagtttgaatt

SEQ ID NO: 451 - DNA, 18 bp, Artificial Sequence
  Oligonucleotide with nucleotide sequence identical to and specific for bisulfite converted methylated and/or non-methylated sense strand DNA of the SNCA(-926/-483) region of the human SNCA gene.
ttgtttgagtttgaatta

SEQ ID NO: 452 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
taattcaaactcaaacaa

SEQ ID NO: 453 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
aattcaaactcaaacaaa

SEQ ID NO: 454 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
attcaaactcaaacaaac

SEQ ID NO: 455 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
ttcaaactcaaacaaaca

SEQ ID NO: 456 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
tcaaactcaaacaaacaa

SEQ ID NO: 457 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
caaactcaaacaaacaac

SEQ ID NO: 458 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
aaactcaaacaaacaaca

SEQ ID NO: 459 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
aactcaaacaaacaacaa

SEQ ID NO: 460 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
actcaaacaaacaacaaa

SEQ ID NO: 461 - DNA, 18 bp, Artificial Sequence
  Oligonucleotide with nucleotide sequence specific for bisulfite converted methylated and/or non-methylated DNA of the SNCA(-926/-483) region of the human SNCA gene, and identical to antisense strand.
ctcaaacaaacaacaaac

SEQ ID NO: 462 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcaaacaaacaacaaacc

SEQ ID NO: 463 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaacaaacaacaaaccc

SEQ ID NO: 464 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacaaacaacaaaccca

SEQ ID NO: 465 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaaacaacaaacccaa

SEQ ID NO: 466 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaacaacaaacccaaa

SEQ ID NO: 467 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaacaacaaacccaaat

SEQ ID NO: 468 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacaacaaacccaaata

SEQ ID NO: 469 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaacaaacccaaatat

SEQ ID NO: 470 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaacaaacccaaatata

SEQ ID NO: 471 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacaaacccaaatataa

SEQ ID NO: 472 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaaacccaaatataat

SEQ ID NO: 473 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaacccaaatataata

SEQ ID NO: 474 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaacccaaatataataa

SEQ ID NO: 475 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacccaaatataataat

SEQ ID NO: 476 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacccaaatataataatt

SEQ ID NO: 477 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acccaaatataataattc

SEQ ID NO: 478 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccaaatataataattct

SEQ ID NO: 479 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaaatataataattcta

SEQ ID NO: 480 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaatataataattctaa

SEQ ID NO: 481 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaatataataattctaat

SEQ ID NO: 482 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatataataattctaatc

SEQ ID NO: 483 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atataataattctaatcc

SEQ ID NO: 484 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tataataattctaatcca

SEQ ID NO: 485 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ataataattctaatccat

SEQ ID NO: 486 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taataattctaatccatc

SEQ ID NO: 487 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aataattctaatccatcc

SEQ ID NO: 488 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ataattctaatccatcca

SEQ ID NO: 489 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taattctaatccatccaa

SEQ ID NO: 490 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aattctaatccatccaac

SEQ ID NO: 491 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
attctaatccatccaaca

SEQ ID NO: 492 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttctaatccatccaacat

SEQ ID NO: 493 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctaatccatccaacatc

SEQ ID NO: 494 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctaatccatccaacatcc

SEQ ID NO: 495 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taatccatccaacatcca

SEQ ID NO: 496 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatccatccaacatccac

SEQ ID NO: 497 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atccatccaacatccacc

SEQ ID NO: 498 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccatccaacatccaccc

SEQ ID NO: 499 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccatccaacatccacccc

SEQ ID NO: 500 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
catccaacatccaccccc

SEQ ID NO: 501 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atccaacatccacccccr

SEQ ID NO: 502 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccaacatccacccccrc

SEQ ID NO: 503 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaacatccacccccrcc

SEQ ID NO: 504 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacatccacccccrccc

SEQ ID NO: 505 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacatccacccccrccct

SEQ ID NO: 506 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acatccacccccrccctc

SEQ ID NO: 507 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
catccacccccrccctca

SEQ ID NO: 508 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atccacccccrccctcaa

SEQ ID NO: 509 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccacccccrccctcaac

SEQ ID NO: 510 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccacccccrccctcaact

SEQ ID NO: 511 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacccccrccctcaacta

SEQ ID NO: 512 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acccccrccctcaactat

SEQ ID NO: 513 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccccrccctcaactatc

SEQ ID NO: 514 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccccrccctcaactatct

SEQ ID NO: 515 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccrccctcaactatcta

SEQ ID NO: 516 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccrccctcaactatctac

SEQ ID NO: 517 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crccctcaactatctacc

SEQ ID NO: 518 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rccctcaactatctaccc

SEQ ID NO: 519 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccctcaactatctaccct

SEQ ID NO: 520 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctcaactatctacccta

SEQ ID NO: 521 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcaactatctaccctaa

SEQ ID NO: 522 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcaactatctaccctaaa

SEQ ID NO: 523 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caactatctaccctaaac

SEQ ID NO: 524 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aactatctaccctaaaca

SEQ ID NO: 525 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
actatctaccctaaacaa

SEQ ID NO: 526 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctatctaccctaaacaaa

SEQ ID NO: 527 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tatctaccctaaacaaac

SEQ ID NO: 528 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atctaccctaaacaaacr

SEQ ID NO: 529 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctaccctaaacaaacrc

SEQ ID NO: 530 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctaccctaaacaaacrcc

SEQ ID NO: 531 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taccctaaacaaacrccr

SEQ ID NO: 532 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accctaaacaaacrccrc

SEQ ID NO: 533 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccctaaacaaacrccrct

SEQ ID NO: 534 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctaaacaaacrccrctc

SEQ ID NO: 535 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctaaacaaacrccrctca

SEQ ID NO: 536 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taaacaaacrccrctcac

SEQ ID NO: 537 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacaaacrccrctcaca

SEQ ID NO: 538 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaaacrccrctcacac

SEQ ID NO: 539 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaacrccrctcacact

SEQ ID NO: 540 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaacrccrctcacactc

SEQ ID NO: 541 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacrccrctcacactcr

SEQ ID NO: 542 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacrccrctcacactcrc

SEQ ID NO: 543 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acrccrctcacactcrcr

SEQ ID NO: 544 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crccrctcacactcrcra

SEQ ID NO: 545 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rccrctcacactcrcraa

SEQ ID NO: 546 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccrctcacactcrcraac

SEQ ID NO: 547 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crctcacactcrcraacc

SEQ ID NO: 548 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rctcacactcrcraaccr

SEQ ID NO: 549 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcacactcrcraaccrt

SEQ ID NO: 550 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcacactcrcraaccrtc

SEQ ID NO: 551 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacactcrcraaccrtct

SEQ ID NO: 552 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acactcrcraaccrtctc

SEQ ID NO: 553 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cactcrcraaccrtctcc

SEQ ID NO: 554 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
actcrcraaccrtctcca

SEQ ID NO: 555 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcrcraaccrtctccaa

SEQ ID NO: 556 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcrcraaccrtctccaac

SEQ ID NO: 557 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crcraaccrtctccaacc

SEQ ID NO: 558 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rcraaccrtctccaaccc

SEQ ID NO: 559 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craaccrtctccaacccc

SEQ ID NO: 560 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raaccrtctccaaccccr

SEQ ID NO: 561 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaccrtctccaaccccrc

SEQ ID NO: 562 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accrtctccaaccccrcr

SEQ ID NO: 563 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccrtctccaaccccrcrc

SEQ ID NO: 564 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crtctccaaccccrcrcc

SEQ ID NO: 565 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rtctccaaccccrcrcca

SEQ ID NO: 566 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctccaaccccrcrccaa

SEQ ID NO: 567 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctccaaccccrcrccaac

SEQ ID NO: 568 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccaaccccrcrccaacc

SEQ ID NO: 569 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaaccccrcrccaacca

SEQ ID NO: 570 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaccccrcrccaaccac

SEQ ID NO: 571 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaccccrcrccaaccacc

SEQ ID NO: 572 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accccrcrccaaccaccc

SEQ ID NO: 573 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccccrcrccaaccacccr

SEQ ID NO: 574 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccrcrccaaccacccra

SEQ ID NO: 575 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccrcrccaaccacccraa

SEQ ID NO: 576 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crcrccaaccacccraaa

SEQ ID NO: 577 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rcrccaaccacccraaaa

SEQ ID NO: 578 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crccaaccacccraaaaa

SEQ ID NO: 579 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rccaaccacccraaaaaa

SEQ ID NO: 580 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaaccacccraaaaaac

SEQ ID NO: 581 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaccacccraaaaaacr

SEQ ID NO: 582 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaccacccraaaaaacrt

SEQ ID NO: 583 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accacccraaaaaacrtt

SEQ ID NO: 584 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccacccraaaaaacrttc

SEQ ID NO: 585 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacccraaaaaacrttct

SEQ ID NO: 586 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acccraaaaaacrttctc

SEQ ID NO: 587 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccraaaaaacrttctca

SEQ ID NO: 588 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccraaaaaacrttctcaa

SEQ ID NO: 589 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craaaaaacrttctcaac

SEQ ID NO: 590 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raaaaaacrttctcaacc

SEQ ID NO: 591 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaacrttctcaacct

SEQ ID NO: 592 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaacrttctcaacctc

SEQ ID NO: 593 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacrttctcaacctcc

SEQ ID NO: 594 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacrttctcaacctcca

SEQ ID NO: 595 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacrttctcaacctccac

SEQ ID NO: 596 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acrttctcaacctccacc

SEQ ID NO: 597 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crttctcaacctccaccc

SEQ ID NO: 598 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rttctcaacctccaccct

SEQ ID NO: 599 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttctcaacctccacccta

SEQ ID NO: 600 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctcaacctccaccctaa

SEQ ID NO: 601 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcaacctccaccctaac

SEQ ID NO: 602 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcaacctccaccctaacr

SEQ ID NO: 603 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacctccaccctaacra

SEQ ID NO: 604 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacctccaccctaacraa

SEQ ID NO: 605 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acctccaccctaacraac

SEQ ID NO: 606 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctccaccctaacraacc

SEQ ID NO: 607 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctccaccctaacraaccc

SEQ ID NO: 608 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccaccctaacraacccc

SEQ ID NO: 609 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaccctaacraaccccr

SEQ ID NO: 610 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caccctaacraaccccrc

SEQ ID NO: 611 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accctaacraaccccrcr

SEQ ID NO: 612 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccctaacraaccccrcrc

SEQ ID NO: 613 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctaacraaccccrcrcc

SEQ ID NO: 614 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctaacraaccccrcrcca

SEQ ID NO: 615 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taacraaccccrcrccaa

SEQ ID NO: 616 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacraaccccrcrccaac

SEQ ID NO: 617 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acraaccccrcrccaaca

SEQ ID NO: 618 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craaccccrcrccaacac

SEQ ID NO: 619 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raaccccrcrccaacact

SEQ ID NO: 620 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaccccrcrccaacactt

SEQ ID NO: 621 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accccrcrccaacactta

SEQ ID NO: 622 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccccrcrccaacacttat

SEQ ID NO: 623 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccrcrccaacacttatt

SEQ ID NO: 624 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccrcrccaacacttatta

SEQ ID NO: 625 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crcrccaacacttattaa

SEQ ID NO: 626 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rcrccaacacttattaac

SEQ ID NO: 627 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crccaacacttattaacc

SEQ ID NO: 628 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rccaacacttattaaccc

SEQ ID NO: 629 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaacacttattaacccr

SEQ ID NO: 630 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacacttattaacccrt

SEQ ID NO: 631 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacacttattaacccrtt

SEQ ID NO: 632 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acacttattaacccrtta

SEQ ID NO: 633 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacttattaacccrttac

SEQ ID NO: 634 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acttattaacccrttacc

SEQ ID NO: 635 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttattaacccrttacca

SEQ ID NO: 636 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttattaacccrttaccac

SEQ ID NO: 637 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tattaacccrttaccacc

SEQ ID NO: 638 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
attaacccrttaccacct

SEQ ID NO: 639 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttaacccrttaccaccta

SEQ ID NO: 640 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taacccrttaccacctat

SEQ ID NO: 641 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacccrttaccacctatt

SEQ ID NO: 642 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acccrttaccacctatta

SEQ ID NO: 643 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccrttaccacctattaa

SEQ ID NO: 644 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccrttaccacctattaac

SEQ ID NO: 645 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crttaccacctattaact

SEQ ID NO: 646 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rttaccacctattaactt

SEQ ID NO: 647 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttaccacctattaactta

SEQ ID NO: 648 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taccacctattaacttaa

SEQ ID NO: 649 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accacctattaacttaac

SEQ ID NO: 650 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccacctattaacttaacc

SEQ ID NO: 651 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacctattaacttaacct

SEQ ID NO: 652 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acctattaacttaacctc

SEQ ID NO: 653 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctattaacttaacctcc

SEQ ID NO: 654 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctattaacttaacctcct

SEQ ID NO: 655 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tattaacttaacctcctt

SEQ ID NO: 656 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
attaacttaacctcctta

SEQ ID NO: 657 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttaacttaacctccttac

SEQ ID NO: 658 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taacttaacctccttaca

SEQ ID NO: 659 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacttaacctccttacac

SEQ ID NO: 660 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acttaacctccttacact

SEQ ID NO: 661 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttaacctccttacactt

SEQ ID NO: 662 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttaacctccttacacttc

SEQ ID NO: 663 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taacctccttacacttcc

SEQ ID NO: 664 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacctccttacacttcca

SEQ ID NO: 665 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acctccttacacttccat

SEQ ID NO: 666 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctccttacacttccatt

SEQ ID NO: 667 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctccttacacttccattt

SEQ ID NO: 668 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccttacacttccatttc

SEQ ID NO: 669 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccttacacttccatttca

SEQ ID NO: 670 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttacacttccatttcat

SEQ ID NO: 671 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttacacttccatttcatt

SEQ ID NO: 672 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tacacttccatttcatta

SEQ ID NO: 673 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acacttccatttcattat

SEQ ID NO: 674 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacttccatttcattatt

SEQ ID NO: 675 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acttccatttcattattt

SEQ ID NO: 676 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttccatttcattattta

SEQ ID NO: 677 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttccatttcattatttac

SEQ ID NO: 678 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccatttcattatttacc

SEQ ID NO: 679 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccatttcattatttacca

SEQ ID NO: 680 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
catttcattatttaccaa

SEQ ID NO: 681 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atttcattatttaccaaa

SEQ ID NO: 682 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tttcattatttaccaaac

SEQ ID NO: 683 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttcattatttaccaaacr

SEQ ID NO: 684 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcattatttaccaaacrc

SEQ ID NO: 685 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cattatttaccaaacrca

SEQ ID NO: 686 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
attatttaccaaacrcaa

SEQ ID NO: 687 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttatttaccaaacrcaac

SEQ ID NO: 688 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tatttaccaaacrcaact

SEQ ID NO: 689 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atttaccaaacrcaactt

SEQ ID NO: 690 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tttaccaaacrcaactta

SEQ ID NO: 691 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttaccaaacrcaacttac

SEQ ID NO: 692 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taccaaacrcaacttact

SEQ ID NO: 693 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accaaacrcaacttactt

SEQ ID NO: 694 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaaacrcaacttactta

SEQ ID NO: 695 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaacrcaacttacttac

SEQ ID NO: 696 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacrcaacttacttact

SEQ ID NO: 697 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacrcaacttacttactt

SEQ ID NO: 698 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acrcaacttacttacttt

SEQ ID NO: 699 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crcaacttacttacttta

SEQ ID NO: 700 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rcaacttacttactttac

SEQ ID NO: 701 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacttacttactttaca

SEQ ID NO: 702 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacttacttactttacaa

SEQ ID NO: 703 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acttacttactttacaaa

SEQ ID NO: 704 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttacttactttacaaaa

SEQ ID NO: 705 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttacttactttacaaaaa

SEQ ID NO: 706 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tacttactttacaaaaaa

SEQ ID NO: 707 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acttactttacaaaaaaa

SEQ ID NO: 708 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttactttacaaaaaaaa

SEQ ID NO: 709 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttactttacaaaaaaaaa

SEQ ID NO: 710 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tactttacaaaaaaaaac

SEQ ID NO: 711 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
actttacaaaaaaaaacc

SEQ ID NO: 712 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctttacaaaaaaaaacca

SEQ ID NO: 713 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tttacaaaaaaaaaccaa

SEQ ID NO: 714 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttacaaaaaaaaaccaaa

SEQ ID NO: 715 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tacaaaaaaaaaccaaaa

SEQ ID NO: 716 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaaaaaaaaccaaaaa

SEQ ID NO: 717 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaaaaaaaaccaaaaaa

SEQ ID NO: 718 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaaccaaaaaaa

SEQ ID NO: 719 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaccaaaaaaac

SEQ ID NO: 720 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaccaaaaaaacr

SEQ ID NO: 721 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaccaaaaaaacra

SEQ ID NO: 722 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaccaaaaaaacraa

SEQ ID NO: 723 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaccaaaaaaacraac

SEQ ID NO: 724 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaccaaaaaaacraaca

SEQ ID NO: 725 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaccaaaaaaacraacaa

SEQ ID NO: 726 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accaaaaaaacraacaaa

SEQ ID NO: 727 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaaaaaaacraacaaac

SEQ ID NO: 728 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaaaaaacraacaaaca

SEQ ID NO: 729 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaacraacaaacaa

SEQ ID NO: 730 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaacraacaaacaaa

SEQ ID NO: 731 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaacraacaaacaaat

SEQ ID NO: 732 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacraacaaacaaatt

SEQ ID NO: 733 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacraacaaacaaattt

SEQ ID NO: 734 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacraacaaacaaatttt

SEQ ID NO: 735 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acraacaaacaaatttta

SEQ ID NO: 736 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craacaaacaaattttat

SEQ ID NO: 737 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raacaaacaaattttata

SEQ ID NO: 738 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaaacaaattttatat

SEQ ID NO: 739 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaacaaattttatata

SEQ ID NO: 740 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaacaaattttatataa

SEQ ID NO: 741 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacaaattttatataaa

SEQ ID NO: 742 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaaattttatataaaa

SEQ ID NO: 743 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaattttatataaaaa

SEQ ID NO: 744 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaattttatataaaaaa

SEQ ID NO: 745 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaattttatataaaaaaa

SEQ ID NO: 746 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aattttatataaaaaaaa

SEQ ID NO: 747 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
attttatataaaaaaaaa

SEQ ID NO: 748 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttttatataaaaaaaaaa

SEQ ID NO: 749 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tttatataaaaaaaaaat

SEQ ID NO: 750 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttatataaaaaaaaaatt

SEQ ID NO: 751 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tatataaaaaaaaaatta

SEQ ID NO: 752 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atataaaaaaaaaattaa

SEQ ID NO: 753 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tataaaaaaaaaattaaa

SEQ ID NO: 754 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ataaaaaaaaaattaaat

SEQ ID NO: 755 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taaaaaaaaaattaaata

SEQ ID NO: 756 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaaattaaatac

SEQ ID NO: 757 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaattaaatact

SEQ ID NO: 758 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaattaaatactc

SEQ ID NO: 759 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaattaaatactca

SEQ ID NO: 760 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaattaaatactcac

SEQ ID NO: 761 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaattaaatactcacr

SEQ ID NO: 762 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaattaaatactcacrc

SEQ ID NO: 763 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaattaaatactcacrct

SEQ ID NO: 764 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aattaaatactcacrctc

SEQ ID NO: 765 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
attaaatactcacrctcc

SEQ ID NO: 766 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttaaatactcacrctcca

SEQ ID NO: 767 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taaatactcacrctccat

SEQ ID NO: 768 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taaatactcacrctccat

SEQ ID NO: 769 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaatactcacrctccata

SEQ ID NO: 770 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatactcacrctccataa

SEQ ID NO: 771 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atactcacrctccataaa

SEQ ID NO: 772 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tactcacrctccataaaa

SEQ ID NO: 773 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
actcacrctccataaaac

SEQ ID NO: 774 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcacrctccataaaaca

SEQ ID NO: 775 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcacrctccataaaacat

SEQ ID NO: 776 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cacrctccataaaacatc

SEQ ID NO: 777 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acrctccataaaacatcc

SEQ ID NO: 778 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crctccataaaacatcct

SEQ ID NO: 779 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rctccataaaacatcctc

SEQ ID NO: 780 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctccataaaacatcctcr

SEQ ID NO: 781 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tccataaaacatcctcrc

SEQ ID NO: 782 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccataaaacatcctcrcr

SEQ ID NO: 783 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cataaaacatcctcrcrt

SEQ ID NO: 784 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ataaaacatcctcrcrtt

SEQ ID NO: 785 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taaaacatcctcrcrttt

SEQ ID NO: 786 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacatcctcrcrtttc

SEQ ID NO: 787 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacatcctcrcrtttcc

SEQ ID NO: 788 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacatcctcrcrtttccc

SEQ ID NO: 789 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acatcctcrcrtttcccr

SEQ ID NO: 790 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
catcctcrcrtttcccra

SEQ ID NO: 791 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atcctcrcrtttcccraa

SEQ ID NO: 792 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcctcrcrtttcccraaa

SEQ ID NO: 793 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctcrcrtttcccraaaa

SEQ ID NO: 794 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcrcrtttcccraaaaa

SEQ ID NO: 795 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcrcrtttcccraaaaaa

SEQ ID NO: 796 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crcrtttcccraaaaaaa

SEQ ID NO: 797 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rcrtttcccraaaaaaaa

SEQ ID NO: 798 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
crtttcccraaaaaaaac

SEQ ID NO: 799 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
rtttcccraaaaaaaacr

SEQ ID NO: 800 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tttcccraaaaaaaacra

SEQ ID NO: 801 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttcccraaaaaaaacraa

SEQ ID NO: 802 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcccraaaaaaaacraat

SEQ ID NO: 803 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccraaaaaaaacraatc

SEQ ID NO: 804 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccraaaaaaaacraatcc

SEQ ID NO: 805 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craaaaaaaacraatccc

SEQ ID NO: 806 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raaaaaaaacraatcccr

SEQ ID NO: 807 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaacraatcccra

SEQ ID NO: 808 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaacraatcccraa

SEQ ID NO: 809 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaacraatcccraaa

SEQ ID NO: 810 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaacraatcccraaaa

SEQ ID NO: 811 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacraatcccraaaaa

SEQ ID NO: 812 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacraatcccraaaaaa

SEQ ID NO: 813 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacraatcccraaaaaac

SEQ ID NO: 814 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acraatcccraaaaaaca

SEQ ID NO: 815 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craatcccraaaaaacaa

SEQ ID NO: 816 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raatcccraaaaaacaac

SEQ ID NO: 817 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatcccraaaaaacaacc

SEQ ID NO: 818 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atcccraaaaaacaacct

SEQ ID NO: 819 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcccraaaaaacaaccta

SEQ ID NO: 820 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccraaaaaacaacctaa

SEQ ID NO: 821 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccraaaaaacaacctaat

SEQ ID NO: 822 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craaaaaacaacctaatc

SEQ ID NO: 823 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raaaaaacaacctaatct

SEQ ID NO: 824 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaacaacctaatctc

SEQ ID NO: 825 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaacaacctaatctct

SEQ ID NO: 826 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacaacctaatctctc

SEQ ID NO: 827 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacaacctaatctctca

SEQ ID NO: 828 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaacctaatctctcaa

SEQ ID NO: 829 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaacctaatctctcaac

SEQ ID NO: 830 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacctaatctctcaacc

SEQ ID NO: 831 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacctaatctctcaaccc

SEQ ID NO: 832 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acctaatctctcaaccct

SEQ ID NO: 833 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cctaatctctcaaccctt

SEQ ID NO: 834 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctaatctctcaaccctta

SEQ ID NO: 835 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taatctctcaacccttat

SEQ ID NO: 836 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatctctcaacccttata

SEQ ID NO: 837 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atctctcaacccttataa

SEQ ID NO: 838 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctctcaacccttataaa

SEQ ID NO: 839 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctctcaacccttataaaa

SEQ ID NO: 840 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctcaacccttataaaaa

SEQ ID NO: 841 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctcaacccttataaaaaa

SEQ ID NO: 842 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcaacccttataaaaaaa

SEQ ID NO: 843 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caacccttataaaaaaaa

SEQ ID NO: 844 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacccttataaaaaaaaa

SEQ ID NO: 845 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acccttataaaaaaaaaa

SEQ ID NO: 846 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cccttataaaaaaaaaaa

SEQ ID NO: 847 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccttataaaaaaaaaaat

SEQ ID NO: 848 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
cttataaaaaaaaaaata

SEQ ID NO: 849 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ttataaaaaaaaaaatat

SEQ ID NO: 850 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tataaaaaaaaaaatatc

SEQ ID NO: 851 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ataaaaaaaaaaatatca

SEQ ID NO: 852 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
taaaaaaaaaaatatcaa

SEQ ID NO: 853 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaaaatatcaaa

SEQ ID NO: 854 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaaatatcaaaa

SEQ ID NO: 855 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaaatatcaaaaa

SEQ ID NO: 856 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaaatatcaaaaac

SEQ ID NO: 857 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaaatatcaaaaaca

SEQ ID NO: 858 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaaatatcaaaaacaa

SEQ ID NO: 859 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaatatcaaaaacaaa

SEQ ID NO: 860 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaatatcaaaaacaaaa

SEQ ID NO: 861 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaatatcaaaaacaaaac

SEQ ID NO: 862 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatatcaaaaacaaaacr

SEQ ID NO: 863 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atatcaaaaacaaaacra

SEQ ID NO: 864 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tatcaaaaacaaaacraa

SEQ ID NO: 865 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atcaaaaacaaaacraaa

SEQ ID NO: 866 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcaaaaacaaaacraaaa

SEQ ID NO: 867 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaaaacaaaacraaaac

SEQ ID NO: 868 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaacaaaacraaaacc

SEQ ID NO: 869 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacaaaacraaaacca

SEQ ID NO: 870 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacaaaacraaaaccaa

SEQ ID NO: 871 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacaaaacraaaaccaaa

SEQ ID NO: 872 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acaaaacraaaaccaaat

SEQ ID NO: 873 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaaacraaaaccaaatc

SEQ ID NO: 874 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaacraaaaccaaatca

SEQ ID NO: 875 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaacraaaaccaaatcaa

SEQ ID NO: 876 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aacraaaaccaaatcaaa

SEQ ID NO: 877 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
acraaaaccaaatcaaat

SEQ ID NO: 878 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
craaaaccaaatcaaatc

SEQ ID NO: 879 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
raaaaccaaatcaaatct

SEQ ID NO: 880 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaaccaaatcaaatctc

SEQ ID NO: 881 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaaccaaatcaaatctct

SEQ ID NO: 882 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaccaaatcaaatctctt

SEQ ID NO: 883 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
accaaatcaaatctcttt

SEQ ID NO: 884 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ccaaatcaaatctctttc

SEQ ID NO: 885 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaatcaaatctctttcc

SEQ ID NO: 886 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaatcaaatctctttcct

SEQ ID NO: 887 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatcaaatctctttcctt

SEQ ID NO: 888 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atcaaatctctttcctta

SEQ ID NO: 889 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tcaaatctctttccttaa

SEQ ID NO: 890 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
caaatctctttccttaaa

SEQ ID NO: 891 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aaatctctttccttaaac

SEQ ID NO: 892 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
aatctctttccttaaact

SEQ ID NO: 893 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
atctctttccttaaactc

SEQ ID NO: 894 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctctttccttaaactcc

SEQ ID NO: 895 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctctttccttaaactccc

SEQ ID NO: 896 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tctttccttaaactcccc

SEQ ID NO: 897 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
ctttccttaaactcccca

SEQ ID NO: 898 - DNA, 18 bp, Artificial Sequence
  Oligonucleotide with nucleotide sequence specific for bisulfite  converted methylated and/or non-methylated DNA of the SNCA(-926/-483) region of the human SNCA gene, and identical to antisense strand.
tttccttaaactccccaa

(19) United States
(12) Patent Application Publication
(10) Pub. No.: US 2013/0309674 A1
(43) Pub. Date: Nov. 21, 2013

(54) ANALYSIS OF THE METHYLATION PATTERN OF THE ALPHA-SYNUCLEIN GENE FROM DNA OF PERIPHERAL BLOOD MONOCYTES FOR DIAGNOSING PARKINSON'S DISEASE

(71) Applicants: Ullrich Wuellner, Wachtberg (DE); Ina Schmitt, Schwerte (DE)

(72) Inventors: Ullrich Wuellner, Wachtberg (DE); Ina Schmitt, Schwerte (DE)

(73) Assignee: Rheinische Friedrich-Wilhelms Universitaet Bonn, Bonn (DE)

(21) Appl. No.: 13/981,605

(22) PCT Filed: Jan. 26, 2012
(86) PCT No.: PCT/EP12/51260

§ 371 Date: Jul. 25, 2013

(30) Foreign Application Priority Data
Jan. 26, 2011 (EP) 11152285.0

(51) Int. Cl. C12Q 1/68

(52) U.S. Cl. CPC C12Q 1/6883
USPC 435/6.11

(57) ABSTRACT

A method of using peripheral blood monocytes of a patient to at least one of diagnose and assess a risk of a patient of developing Parkinson's disease includes obtaining peripheral blood monocytes from the patient, and determining a methylation pattern of the SNCA_{(-926/-483)} region of the SNCA gene of the DNA of the peripheral blood monocytes.

### CROSS REFERENCE TO PRIOR APPLICATIONS

This application is a U.S. National Phase application under 35 U.S.C. §371 of International Application No. PCT/EP2012/051260, filed on Jan. 26, 2012 and which claims benefit to European Patent Application No. 11152285.0, filed on Jan. 26, 2011. The International Application was published in English on Aug. 2, 2012 as WO 2012/101228 A1 under PCT Article 21(2).

### FIELD

The present invention relates to the diagnosis of Parkinson's disease, especially to the diagnosis of sporadic Parkinson's disease. The present invention relates to the use of peripheral blood monocytes for determining the methylation status of the human alpha-synuclein gene for diagnosing Parkinson's disease and/or for assessing a patient's risk of developing Parkinson's disease. The present invention further provides a method for determining methylation of the human alpha-synuclein gene in peripheral blood monocytes for diagnosing Parkinson's disease and/or for assessing a patient's risk of developing Parkinson's disease. The present invention also provides a method for diagnosing Parkinson's disease and/or for assessing a patient's risk of developing Parkinson's disease

### BACKGROUND

Parkinson's disease (“PD”) is one of the most common neurodegenerative diseases. Approximately 2% of elderly persons (older than 65) are affected. The clinical symptoms of PD are characterized by akinetic-rigid motor dysfunction and cognitive, affective and neurobehavioural impairments. The most prominent neuropathological characteristics of Parkinson's disease are progressive cell death within the nigro-striatal dopaminergic system, and the appearance of cytoplasmic protein aggregates, the so-called Lewy bodies, in cells of the central nervous system of affected persons.

The majority of PD cases occur sporadically, and its etiology is largely unknown. Pursuant to recent findings, PD is usually diagnosed in an advanced stage of the disease. A neuro-protective or neuro-restorative therapy can therefore only be launched late in the disease's progression. Current methods of diagnosing PD, for example, single photon emission computed tomography (SPECT) or positron emission tomography (PET), are expensive and are therefore only used subsequent to clinical presumption diagnosis or for differential diagnosis. Although some symptoms such as sensory and sleep difficulties are discussed as useful in early diagnosis of PD, they are not specific enough and lack sensitivity. A simple to perform and inexpensive test for determining the risk of being affected by PD is not yet available.

According to its pathophysiology, PD may be considered a synucleinopathy due to the abnormal accumulation of the alpha-synuclein (SNCA) protein in the brain of patients having developed PD. Since the alpha-synuclein protein is the main component of Lewy bodies, a number of specific genetic mutations of the alpha-synuclein gene associated with PD have been discovered. Missense mutations (mutation in which a single nucleotide is changed) of the gene, and duplications and triplications of the locus containing it have been found in different groups with familial PD. Missense mutations are very rare with few families found in all the world. On the other hand, multiplications of the SNCA locus account for around 2% of familial cases. Multiplications have additionally been found in non symptomatic carriers leading to the conclusion that penetrance is incomplete or age-dependent.

A fragment of the alpha-synuclein is known to be the non-Abeta component of Alzheimer's disease amyloid. Said fragment was originally found in an amyloid-enriched fraction, and identified as a fragment of its precursor protein, NCAP, by cloning of the full-length cDNA. It was later determined that NCAP is the human homologue of Torpedo synuclein. NCAP is now therefore referred to as human alpha-synuclein.

It recently became evident that SNCA expression is an important factor in pathogenesis of PD as increased gene dosage of SNCA leads to familial PD, and polymorphisms in the SNCA promoter increase the risk of developing sporadic PD. SNCA mRNA levels were furthermore found to be increased in individual laser-captured dopaminergic neurons in substantia nigra of PD patients. It seems that already a minor increase of wild-type SNCA protein in the presynaptic compartment of neurons is sufficient to induce dysfunction and degeneration.

Voustinas and colleagues have studied the expression of alleles of the alpha-synuclein gene in a lymphoblastoid cell line and in blood cells of a patient heterozygous for the mutation p.Ala53Thr, the first mutation implicated in PD pathogenesis (Voustinas, G. E. et al. (2010), Allelic imbalance of expression and epigenetic regulation within the alpha-synuclein wild-type and p.Ala53Thr alleles in Parkinson disease, Hum. Mutat. 31(6):685-691). Said patient was diagnosed with early-onset PD, and belonging to a family with multiple PD cases in several generations, with autosomal dominant inheritance and <100% penetrance of the disease. Evidence is provided that SNCA shows monoalleleic expression in this patient, that epigenetic silencing of the mutated allele involves histone modifications but not DNA methylation, because expression of the mutant allele was silenced despite the finding that all analyzed CpG sites were found to be unmethylated, and that steady state mRNA levels deriving from the normal SNCA allele in this patient exceeded those of both normal SNCA alleles in matching control individuals. An imbalanced SNCA expression has thus been documented, with silencing the expression of the p.Ala53Thr allele and upregulation of the expression of the wild-type allele.

Grundemann and colleagues detected significantly elevated alpha-synuclein mRNA levels in individual, surviving neuromelanin- and tyrosine hydroxylase-positive substantia nigra dopaminergic neurons from idiopathic PD brains compared to unaffected controls (Grundemann, J. et al. (2008), Elevated a-synuclein mRNA levels in individual UV-laser-microdissected dopaminergic substantia nigra neurons in idiopathic Parkinson's disease, NAR 36(7): e38). This data demonstrate a cell-type specific transcriptional upregulation of alpha-synuclein expression in neurons being a key population in PD.

It has also been found in in-vitro studies that methylation of human SNCA intron I decreased gene expression, while inhibition of DNA methylation activated SNCA expression. Notably, methylation of SNCA intron I was reduced in DNA from sporadic PD patient's substantia nigra putamen, and cortex (Jowaed, A. et al. (2010), Methylation regulates alpha-synuclein expression and is decreased in Parkinson's disease patients' brains. J. Neurosci, 30(18):6355-6359). Methylation pattern in the SNCA region of interest varied considerably, however, between tissues and individuals.

Besides of its association with Alzheimer's disease and Parkinson's disease, epigenetic regulation of the SNCA gene's expression appears to also be involved in other disorders. For example, an increased methylation of the alpha synuclein promoter DNA in patients with alcoholism was observed (Bonsch, D. et al. (2005), DNA hypermethylation of the alpha synuclein promoter in patients with alcoholism, Neuroreport. 8; 16(2):167-170). The increased methylation of the alpha synuclein promoter DNA was significantly associated with elevated homocysteine levels in said patients. Since hypermethylation of DNA is an important epigenetic factor in down regulation of gene expression, and since alpha synuclein has been linked to craving, hypermethylation of alpha-synuclein promoter DNA was discussed to explain the reduced value of craving under alcohol drinking conditions. In contrast, alpha-synuclein protein expression was found to be significantly higher in patients with alcoholism when compared to healthy controls (Bonsch, D. et al. (2005), Alpha-synuclein protein levels are increased in alcoholic patients and are linked to craving, Alcohol Clin. Res. 29(5):763-765).

Global DNA hypomethylation and DNA hypermethylation of the alpha-synuclein promoter was furthermore observed in females with anorexia nervosa indicating epigenetic contribution to the pathophysiology of eating disorders. In patients with anorexia, a decreased alpha-synuclein expression was associated with DNA hypermethylation of the alpha-synuclein promoter. (Frieling, H. et al. (2007), Global DNA hypomethylation and DNA hypermethylation of the alpha synuclein promoter in females with anorexia nervosa, Mol. Psychiatry 12:229-230).

### SUMMARY

In an embodiment, the present invention provides a method of using peripheral blood monocytes of a patient to at least one of diagnose and assess a risk of a patient of developing Parkinson's disease which includes obtaining peripheral blood monocytes from the patient, and determining a methylation pattern of the SNCA(−926/−483) region of the SNCA gene of the DNA of the peripheral blood monocytes.

### BRIEF DESCRIPTION OF THE DRAWINGS

The present invention is described in greater detail below on the basis of embodiments and of the drawings in which:

FIG. 1 shows the nucleotide sequence of a region (SNCA_{(-926,-483)}) within the first intron of the human SNCA gene and shows the predicted transcription factor binding sites adjacent to the CpG dinucleotides within SNCA_{(-926/-483)}; and

FIG. 2 shows the methylation pattern of the SNCA_{(-926,-483)} region of the human SNCA gene in peripheral blood monocytes of Parkinson patients (black columns) by displaying the percentage of methylated cysteine residues at each CpG dinucleotide in said region compared to neurologically healthy individuals (white columns).

### SEQUENCE LISTING

The Sequence Listing associated with this application is filed in electronic form via EFS-Web and is hereby incorporated by reference into this specification in its entirety. The name of the text file containing the Sequence Listing is Sequence_Listing_{—}18072013. The size of the text file is 357,318 Bytes, and the text file was created on Jul. 18, 2013.

### DETAILED DESCRIPTION

The present invention is based on the surprising findings that a decreased methylation in the 5′ region of the human SNCA gene, which was observed in DNA from cells of the central nervous system of patients having developed Parkinson's disease, can also be found in the DNA obtained from peripheral blood monocytes of patients having developed Parkinson's disease. These findings permit providing an inexpensive and easy to perform test for diagnosis of Parkinson's disease. Such a test is further advantageous, because it permits diagnosis of Parkinson's disease at a very early stage of the disease's progression and may even permit assessing a person's risk of developing Parkinson's disease when becoming older, even if no symptoms are yet present.

In an embodiment, the present invention provides for the use of peripheral blood monocytes of a patient for diagnosis of Parkinson's disease and/or assessing the patient's risk of developing Parkinson's disease, wherein the methylation pattern of the SNCA gene from DNA of said peripheral blood monocytes is determined.

In an embodiment, the present invention provides a method of determining the methylation pattern of the SNCA gene from DNA of peripheral blood monocytes of a patient for diagnosis of Parkinson's disease and/or assessing the patient's risk of developing Parkinson's disease.

In an embodiment, the present invention provides a method for diagnosis and/or assessing a patient's risk of developing Parkinson's disease, wherein the methylation pattern of the SNCA gene from DNA of peripheral blood monocytes of said patient is determined.

The term “patient” refers to any person whose risk of developing Parkinson's disease during his or her lifetime is to be assessed or who is to be diagnosed for Parkinson's disease, regardless of whether clinical symptoms, in particular neurological symptoms being associated with Parkinson's disease, are present or not. The term “patient” is therefore not restricted to persons already affected by Parkinson's disease, but also refers to healthy individuals.

In an embodiment of the present invention, peripheral blood monocytes of a patient are used for diagnosis of whether the patient has developed Parkinson's disease and/or for assessing the patient's risk of developing Parkinson's disease, even if said patient does not shown any neurological symptoms associated with Parkinson's disease. For diagnosis of whether the patient has developed Parkinson's disease and/or for assessing the patient's risk of developing Parkinson's disease, in particular sporadic PD, the methylation pattern of the SNCA gene obtained from genomic DNA of the patient's peripheral blood monocytes is determined.

Using peripheral blood monocytes for diagnosis of whether the patient has developed Parkinson's disease and/or for assessing the patient's risk of developing Parkinson's disease is advantageous over methods currently used for diagnosing Parkinson's disease because peripheral blood monocytes of a patient can be easily obtained without posing undue health hazards to the patient and without the need of expensive technical equipment. Peripheral blood monocytes can moreover be used even if the patient does not suffer from neurological symptoms associated with Parkinson's disease. The present invention therefore provides an opportunity to determine whether a person is at risk of developing PD in the future. It may also be possible to use peripheral blood monocytes from a patient's blood sample that was taken for other reasons than the instant diagnosis or risk assessment. For example, using peripheral blood monocytes from blood samples of a patient that were taken at various points of time during the patient's life, alterations in the methylation pattern of the SNCA gene can be monitored without extensive costs such that the etiology of sporadically occurring PD may be determined.

In an embodiment of the present invention, the use of peripheral blood monocytes of a patient for diagnosing and/or assessing the patient's risk of developing Parkinson's disease is characterized in that the methylation pattern of intron 1 of the SNCA gene from genomic DNA of the patient's peripheral blood monocytes is determined.

In an embodiment of the present invention, the use of peripheral blood monocytes of a patient for diagnosing and/or assessing the patient's risk of developing Parkinson's disease is characterized in that the methylation pattern of the SNCA_{(-926,-483)} region of the human SNCA gene from genomic DNA of the patient's peripheral blood monocytes is determined.

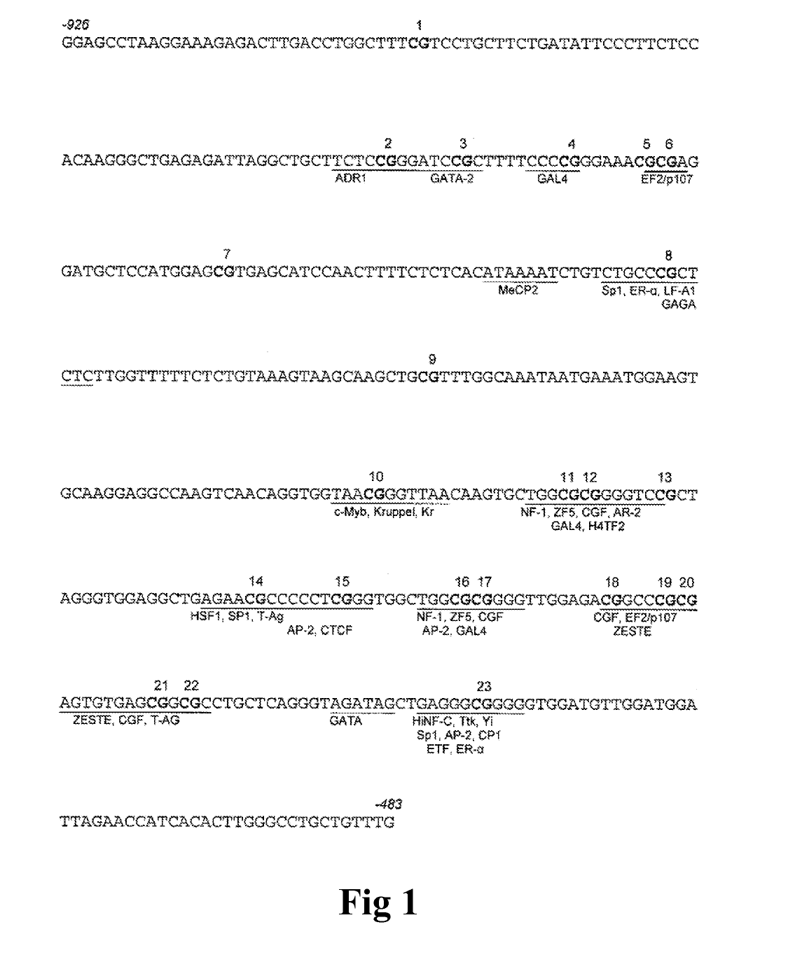

The SNCA_{(-926,-483)} region of the SNCA gene is the nucleotide sequence of the SNCA gene upstream of the ATG start codon for SNCA, consisting of the nucleotides sequence from nucleotide −926 to nucleotide −483 with respect to the ATG start codon of SNCA, the adenosine (A) of said start codon being designated nucleotide 1. The SNCA_{(-926/-483)} region is therefore located upstream or 5′ of the start codon as is identified by the minus in front of the designated position numbers. Notably, intron 1 of the human SNCA gene and hence the SNCA_{(-926,-483)} region of the human SNCA gene does not comprise the promoter of the human SNCA gene. The promoter of the human SNCA gene is located upstream, i.e., in 5′ direction, of exon 1 which in turn is located upstream of and adjacent to said intron 1. The nucleotide sequence of SNCA_{(-926,-483)} region (SEQ ID NO: 1) of the human SNCA gene is shown in FIG. 1.

The SNCA_{(-926,-483)} region of the human SNCA gene comprises 23 CpG dinucleotides, wherein the cytosine of each of these CpG dinucleotides may or may not be methylated. Whether the cytosine of a given CpG dinucleotide is methylated or not defines the methylation status of the cytosine of said particular CpG dinucleotide. The methylation pattern of a DNA molecule or a region of a DNA molecule, for example, a gene such as the SNCA gene, or of the SNCA_{(-926,-483)} region of the human SNCA gene, is defined by the combination of the methylation status of each CpG dinucleotide within said DNA molecule or said region. Table 1 provides an overview of the 23 CpG dinucleotides within the SNCA_{(-926/-483)} region of the human SNCA gene and their position within intron 1 with respect to the ATG start codon of the human SNCA gene.

| No. | Position |
|---|---|
| 1 | −894/−893 |
| 2 | −838/−837 |
| 3 | −831/−830 |
| 4 | −821/−820 |
| 5 | −814/−813 |
| 6 | −812/−811 |
| 7 | −794/−793 |
| 8 | −753/−752 |
| 9 | −716/−715 |
| 10 | −662/−661 |
| 11 | −644/−643 |
| 12 | −642/−641 |
| 13 | −635/−634 |
| 14 | −614/−613 |
| 15 | −606/−605 |
| 16 | −595/−594 |
| 17 | −593/−592 |
| 18 | −581/−580 |
| 19 | −576/−575 |
| 20 | −574/−573 |
| 21 | −564/−563 |
| 22 | −561/−560 |
| 23 | −534/−533 |

Table 1: List of the 23 CpG dinucleotides within the SNCA_{(-926/-483)} region of the human SNCA gene which may be subject to methylation. The position of each of these 23 CpG dinucleotides with reference to the ATG start codon of the human SNCA gene is provided, said start codon consisting of nucleotides 1 to 3 pursuant to the nomenclature used herein.

In an embodiment of the present invention, the methylation status of one or more CpG dinucleotides of the SNCA_{(-926,-483)} region of the human SNCA gene is determined. Said one or more CpG dinucleotides can, for example, be selected from the group consisting of CpG dinucleotides No. 1, 2, 7, 8, 11, 12, 13, 18, 21, 22 and 23 of the SNCA_{(-926,-483)} region of the human SNCA gene. Said one or more CpG dinucleotides can, for example, be selected from the group consisting of CpG dinucleotides No. 1, 7, 8, 13, 22 and 23 of the SNCA_{(-926/-483)} region of the human SNCA gene.

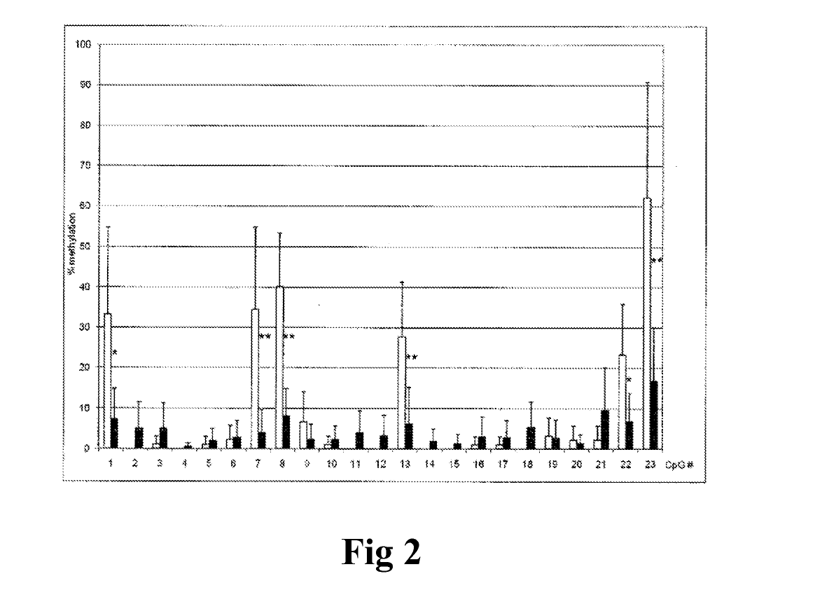

CpG dinucleotides No. 1, 7, 8, 13, 22 and 23 of the SNCA_{(-926,-483)} region of the human SNCA gene can, for example, be selected because it was assessed that the methylation status of these particular CpG dinucleotides is significantly altered in patients suffering from Parkinson's disease, in particular from sporadic PD, compared to healthy control individuals. More specifically, the methylation pattern of the SNCA_{(-926,-483)} region of the human SNCA gene from peripheral blood monocytes as displayed in FIG. 2 illustrates that the methylation status of CpG dinucleotides No. 1, 7, 8, 13, 22 and 23 is significantly altered in patients suffering from sporadic PD. In said patients, the percentage of methylated cytosines in at least one of CpG dinucleotides No. 1, 7, 8, 13, 22 and 23 is significantly reduced compared to the control DNA from healthy individuals. The SNCA_{(-926,-483)} region of the human SNCA gene from peripheral blood monocytes is therefore hypomethylated in PD patients affected with sporadic PD. The SNCA_{(-926/-483)} region of the human SNCA gene from peripheral blood monocytes is hypomethylated with respect to its CpG dinucleotides No. 1, 7, 8, 13, 22 and 23.

In addition, the percentage of methylated CpG dinucleotide No. 2, 11, 12, 18 and 21 of the SNCA_{(-926,-483)} region of the human SNCA gene from peripheral blood monocytes appears to be increased in patients affected with sporadic PD. The evidence is striking although at present not statistically significant due to the low number of probes that were thus far analyzed. However, the methylation pattern of the SNCA_{(-926,-483)} region of the human SNCA gene from peripheral blood monocytes is altered in patients affected by PD in that said methylation pattern displays a hypermethylation with respect to CpG dinucleotide No. 2, 11, 12, 18 and 21 of the SNCA_{(-926,-483)} region of the human SNCA gene from peripheral blood monocytes.

Notwithstanding that virtually any method for analyzing the methylation status of a given CpG dinucleotide can be employed for analyzing the methylation status of the human SNCA gene, of intron 1 of the human SNCA gene, and/or of the SNCA_{(-926,-483)} region of the human SNCA gene, in particular of one or more CpG dinucleotides of the SNCA_{(-926,-483)} region of the human SNCA gene, for example, of one or more CpG dinculeotides of the SNCA_{(-926,-483)} region of the human SNCA gene selected from the group consisting of CpG dinucleotides No. 1, 2, 7, 8, 11, 12, 13, 18, 21, 22 and 23 of the SNCA_{(-926,-483)} region of the human SNCA gene, and, for example, of one or more CpG dinucleotides selected from the group consisting of CpG dinucleotides No. 1, 7, 8, 13, 22 and 23 of the SNCA_{(-926,-483)} region of the human SNCA gene, the methylation status can, for example, be determined by one or more methods selected from the group consisting of methylation specific PCR, direct sequence analysis of bisulfite-treated DNA, COBRA-Assay and analysis of methylation-specific restriction enzyme digestion.

In an embodiment of the present invention, the methylation pattern of the human SNCA-gene, of intron 1 of the human SNCA gene, or of the SNCA_{(-926,-483)} region of the human SNCA gene, in particular of one or more CpG dinucleotides of the SNCA_{(-926,-483)} region of the human SNCA gene, for example, of one or more CpG dinucleotides of the SNCA_{(-926,-483)} region of the human SNCA gene selected from the group consisting of CpG dinucleotides 1, 2, 7, 8, 11, 12, 13, 18, 21, 22 and 23 of the SNCA_{(-926/-483)} region of the human SNCA gene is determined by methylation specific PCR (MSP), and, for example, of one or more CpG dinucleotides selected from the group consisting of CpG dinucleotides No. 1, 7, 8, 13, 22 and 23 of the SNCA_{(-926/-483)} region of the human SNCA gene.

In animals, DNA methylation predominantly involves the addition of a methyl group to the carbon-5 position of cytosine residues of the dinucleotide CpG, and is implicated in repression of transcriptional activity. Treatment of DNA with sodium bisulfite converts cytosine residues to uracil, but leaves 5-methylcytosine residues unaffected. Sodium bisulfite treatment thus introduces specific changes in the DNA sequence that depend on the methylation status of individual cytosine residues, yielding single-nucleotide resolution information about the methylation status of a segment of DNA.

MSP can rapidly assess the methylation status of virtually any group of CpG dinucleotides within a CpG island, independent of the use of cloning or methylation-sensitive restriction enzymes. The MSP comprises initial modification of DNA by sodium bisulfite, converting all unmethylated, but not methylated, cytosines to uracil, and subsequent amplification with primers specific for methylated versus unmethylated DNA. MSP requires very small quantities of DNA, and is sensitive to 0.1% methylated alleles of a given CpG island locus.

In an embodiment of the present invention, methylation of the human SNCA-gene, of intron 1 of the human SNCA gene, and/or of the SNCA_{(-926,-483)} region of the human SNCA gene, in particular of one or more CpG dinucleotides of the SNCA_{(-926,-483)} region of the human SNCA gene, for example, of one or more CpG dinculeotides of the SNCA_{(-926,-483)} region of the human SNCA gene selected from the group consisting of CpG dinucleotides 1, 2, 7, 8, 11, 12, 13, 18, 21, 22 and 23 of the SNCA_{(-926,-483)} region of the human SNCA gene and, for example, of one or more CpG dinucleotides selected from the group consisting of CpG dinucleotides No. 1, 7, 8, 13, 22 and 23 of the SNCA_{(-926,-483)} region of the human SNCA gene is determined by sequence analysis of bisulfite treated DNA.

The method of methylation analysis utilizing sequence analysis of bisulfite-treated DNA involves bisulfite treatment of the DNA to be analyzed, PCR amplification of the bisulfite treated DNA, cloning of the amplification product, and standard dideoxynucleotide DNA sequencing of the cloned DNA fragments to directly determine the nucleotides resistant to bisulfite conversion. Primers for PCR amplification can, for example, be designed to be strand-specific but not methylation-specific, i.e., the nucleotide sequence of the preferred primers do not comprise a sequence corresponding to a nucleotide sequence including a CpG dinucleotide. The aforementioned primers for PCR amplification flank do not, for example, involve the methylation site or methylation sites of interest. However, in an additional or alternative embodiment, at least one or both, the forward primer and the reverse primer for PCR amplification may cover one or more CpG dinucleotides. To be strand specific and to allow amplification of methylated as well as non-methylated DNA fragments, a mixture of primers may be utilized, wherein the mixture of strand primers consists of primers having a pyrimidine nucleotide (Y) for the cytosine of the CpG dinucleotide within the DNA fragment to be amplified and covered by said strand primer, i.e., a C for amplification of said DNA fragment which is methylated at said C, or a T for amplification of said DNA fragment which is not methylated at said C. The PCR amplification will therefore amplify both methylated and unmethylated sequences, in contrast to methylation-specific PCR. All sites of unmethylated cytosines are displayed as thymines in the resulting amplified sequence of the sense strand, and as adenines in the amplified antisense strand. This method requires cloning of the PCR products prior to sequencing for adequate sensitivity. Alternatively, nested PCR methods can be used to enhance the product for sequencing.

Pyrosequencing may also be used to analyze bisulfite-treated DNA without using methylation-specific PCR. Following PCR amplification of the region of interest, pyrosequencing is used to determine the bisulfite-converted sequence of specific CpG sites in the region. The ratio of C-to-T at individual sites can be determined quantitatively based on the amount of C and T incorporation during the sequence extension.

In an embodiment of the present invention, methylation of the human SNCA-gene, of intron 1 of the human SNCA gene, or of the SNCA_{(-926,-483)} region of the human SNCA gene, in particular of one or more CpG dinucleotides of the SNCA_{(-926,-483)} region of the human SNCA gene, for example, of one or more CpG dinculeotides of the SNCA_{(-926,-483)} region of the human SNCA gene selected from the group consisting of CpG dinucleotides No. 1, 2, 7, 8, 11, 12, 13, 18, 21, 22 and 23 of the SNCA_{(-926,-483)} region of the human SNCA gene, and, for example, of one or more CpG dinucleotides selected from the group consisting of CpG dinucleotides No. 1, 7, 8, 13, 22 and 23 of the SNCA_{(-926,-483)} region of the human SNCA gene, is determined by combined bisulfite restriction analysis (COBRA assay).

The COBRA assay is a quantitative technique to determine DNA methylation levels at specific gene loci in small amounts of genomic DNA. In the COBRA assay, restriction enzyme digestion is used to reveal methylation dependent sequence differences in PCR products of sodium bisulfite-treated DNA. Methylation levels in the original DNA sample are represented by the relative amounts of digested and undigested PCR product in a linearly quantitative fashion across a wide spectrum of DNA methylation levels. The COBRA assay is easy to use and provides quantitative accuracy.

In the COBRA assay, methylation-dependent sequence differences are introduced into the genomic DNA by sodium bisulfite treatment and subsequent PCR amplification of the bisulfite treated DNA. This combination of bisulfite treatment and PCR amplification results in conversion of unmethylated cytosine residues to thymine and of methylated cytosine residues to cytosine. This sequence conversion can lead to methylation dependent creation of new restriction enzyme sites or it can lead to the methylation dependent retention of pre-existing restriction enzyme sites such as, for example, BstUI (CGCG). The primers used in the PCR amplification reaction do not contain CpG dinucleotides so that the amplification step does not discriminate between templates according to their original methylation status. Therefore, in the mixed population of DNA fragments resulting from said PCR, the fraction that has a newly created or retained restriction site that contains a CpG(s) directly reflects the percentage DNA methylation at that site in the original genomic DNA.

The aforementioned methods, i.e., MSP, direct sequencing of bisulfate-treated DNA, and COBRA, utilize oligonucleotides for analyzing the methylation pattern of a DNA fragment, for example, as primers in PCR amplification reactions. For analyzing the methylation pattern of the human SNCA gene, of intron 1 of the human SNCA gene, or of the of the SNCA_{(-926,-483)} region of the human SNCA gene, for example, of one or more CpG dinucleotides of the SNCA_{(-926,-483)} region of the human SNCA gene selected from the group consisting of CpG dinucleotides No. 1, 2, 7, 8, 11, 12, 13, 18, 21, 22 and 23 of the SNCA_{(-926/-483)} region of the human SNCA gene, and, for example, of one or more CpG dinucleotides selected from the group consisting of CpG dinucleotides No. 1, 7, 8, 13, 22 and 23 of the SNCA_{(-926/-483)} region of the human SNCA gene, one or more oligonucleotides comprising a nucleotide sequence selected from the group consisting of SEQ ID NO: 5 to SEQ ID NO: 898 may be used.

Any one of said oligonucleotides comprising a nucleotide sequence selected from the group consisting of SEQ ID NO: 5 to SEQ ID NO: 898 may consist of 16, 17, 18, 19, 20, 21, 22 or more nucleotides. Oligonucleotides for use in analyzing the methylation pattern of the human SNCA gene, of intron 1 of the human SNCA gene, or of the of the SNCA_{(-926,-483)} region of the human SNCA gene, for example, of one or more CpG dinculeotides of the SNCA_{(-926,-483)} region of the human SNCA gene selected from the group consisting of CpG dinucleotides No. 1, 2, 7, 8, 11, 12, 13, 18, 21, 22 and 23 of the SNCA_{(-926,-483)} region of the human SNCA gene, and, for example, of one or more CpG dinucleotides selected from the group consisting of CpG dinucleotides No. 1, 7, 8, 13, 22 and 23 of the SNCA_{(-926,-483)} region of the human SNCA gene, can, for example, consist of 18 nucleotides having a nucleotide sequences selected from the group consisting of SEQ ID NO: 5 to SEQ ID NO: 898.

| | SEQ ID |
|---|---|
| Sequence | NO: |
| T T G G G G A G T T T A A G G A A A | 5 |
| T G G G G A G T T T A A G G A A A G | 6 |
| G G G G A G T T T A A G G A A A G A | 7 |
| G G G A G T T T A A G G A A A G A G | 8 |
| G G A G T T T A A G G A A A G A G A | 9 |
| G A G T T T A A G G A A A G A G A T | 10 |
| A G T T T A A G G A A A G A G A T T | 11 |
| G T T T A A G G A A A G A G A T T T | 12 |
| T T T A A G G A A A G A G A T T T G | 13 |
| T T A A G G A A A G A G A T T T G A | 14 |
| T A A G G A A A G A G A T T T G A T | 15 |
| A A G G A A A G A G A T T T G A T T | 16 |
| A G G A A A G A G A T T T G A T T T | 17 |
| G G A A A G A G A T T T G A T T T G | 18 |
| G A A A G A G A T T T G A T T T G G | 19 |
| A A A G A G A T T T G A T T T G G T | 20 |
| A A G A G A T T T G A T T T G G T T | 21 |
| A G A G A T T T G A T T T G G T T T | 22 |
| G A G A T T T G A T T T G G T T T T | 23 |
| A G A T T T G A T T T G G T T T T Y | 24 |
| G A T T T G A T T T G G T T T T Y G | 25 |
| A T T T G A T T T G G T T T T Y G T | 26 |
| T T T G A T T T G G T T T T Y G T T | 27 |
| T T G A T T T G G T T T T Y G T T T | 28 |
| T G A T T T G G T T T T Y G T T T T | 29 |
| G A T T T G G T T T T Y G T T T T G | 30 |
| A T T T G G T T T T Y G T T T T G T | 31 |
| T T T G G T T T T Y G T T T T G T T | 32 |
| T T G G T T T T Y G T T T T G T T T | 33 |
| T G G T T T T Y G T T T T G T T T T | 34 |
| G G T T T T Y G T T T T G T T T T T | 35 |
| G T T T T Y G T T T T G T T T T T G | 36 |
| T T T T Y G T T T T G T T T T T G A | 37 |
| T T T Y G T T T T G T T T T T G A T | 38 |
| T T Y G T T T T G T T T T T G A T A | 39 |
| T Y G T T T T G T T T T T G A T A T | 40 |
| Y G T T T T G T T T T T G A T A T T | 41 |
| G T T T T G T T T T T G A T A T T T | 42 |
| T T T T G T T T T T G A T A T T T T | 43 |
| T T T G T T T T T G A T A T T T T T | 44 |
| T T G T T T T T G A T A T T T T T T | 45 |
| T G T T T T T G A T A T T T T T T T | 46 |
| G T T T T T G A T A T T T T T T T T | 47 |
| T T T T T G A T A T T T T T T T T T | 48 |
| T T T T G A T A T T T T T T T T T T | 49 |
| T T T G A T A T T T T T T T T T T T | 50 |
| T T G A T A T T T T T T T T T T T A | 51 |
| T G A T A T T T T T T T T T T T A T | 52 |
| G A T A T T T T T T T T T T T A T A | 53 |
| A T A T T T T T T T T T T T A T A A | 54 |
| T A T T T T T T T T T T T A T A A G | 55 |
| A T T T T T T T T T T T A T A A G G | 56 |
| T T T T T T T T T T T A T A A G G G | 57 |
| T T T T T T T T T T A T A A G G G T | 58 |
| T T T T T T T T T A T A A G G G T T | 59 |
| T T T T T T T T A T A A G G G T T G | 60 |
| T T T T T T T A T A A G G G T T G A | 61 |
| T T T T T T A T A A G G G T T G A G | 62 |
| T T T T T A T A A G G G T T G A G A | 63 |
| T T T T A T A A G G G T T G A G A G | 64 |
| T T T A T A A G G G T T G A G A G A | 65 |
| T T A T A A G G G T T G A G A G A T | 66 |
| T A T A A G G G T T G A G A G A T T | 67 |
| A T A A G G G T T G A G A G A T T A | 68 |
| T A A G G G T T G A G A G A T T A G | 69 |
| A A G G G T T G A G A G A T T A G G | 70 |
| A G G G T T G A G A G A T T A G G T | 71 |
| G G G T T G A G A G A T T A G G T T | 72 |
| G G T T G A G A G A T T A G G T T G | 73 |
| G T T G A G A G A T T A G G T T G T | 74 |
| T T G A G A G A T T A G G T T G T T | 75 |
| T G A G A G A T T A G G T T G T T T | 76 |
| G A G A G A T T A G G T T G T T T T | 77 |
| A G A G A T T A G G T T G T T T T T | 78 |
| G A G A T T A G G T T G T T T T T T | 79 |
| A G A T T A G G T T G T T T T T T Y | 80 |
| G A T T A G G T T G T T T T T T Y G | 81 |
| A T T A G G T T G T T T T T T Y G G | 82 |
| T T A G G T T G T T T T T T Y G G G | 83 |
| T A G G T T G T T T T T T Y G G G A | 84 |
| A G G T T G T T T T T T Y G G G A T | 85 |
| G G T T G T T T T T T Y G G G A T T | 86 |
| G T T G T T T T T T Y G G G A T T Y | 87 |
| T T G T T T T T T Y G G G A T T Y G | 88 |
| T G T T T T T T Y G G G A T T Y G T | 89 |
| G T T T T T T Y G G G A T T Y G T T | 90 |
| T T T T T T Y G G G A T T Y G T T T | 91 |
| T T T T T Y G G G A T T Y G T T T T | 92 |
| T T T T Y G G G A T T Y G T T T T T | 93 |
| T T T Y G G G A T T Y G T T T T T T | 94 |
| T T Y G G G A T T Y G T T T T T T T | 95 |
| T Y G G G A T T Y G T T T T T T T T | 96 |
| Y G G G A T T Y G T T T T T T T T Y | 97 |
| G G G A T T Y G T T T T T T T T Y G | 98 |
| G G A T T Y G T T T T T T T T Y G G | 99 |
| G A T T Y G T T T T T T T T Y G G G | 100 |
| A T T Y G T T T T T T T T Y G G G A | 101 |
| T T Y G T T T T T T T T Y G G G A A | 102 |
| T Y G T T T T T T T T Y G G G A A A | 103 |
| Y G T T T T T T T T Y G G G A A A Y | 104 |
| G T T T T T T T T Y G G G A A A Y G | 105 |
| T T T T T T T T Y G G G A A A Y G Y | 106 |
| T T T T T T T Y G G G A A A Y G Y G | 107 |
| T T T T T T Y G G G A A A Y G Y G A | 108 |
| T T T T T Y G G G A A A Y G Y G A G | 109 |
| T T T T Y G G G A A A Y G Y G A G G | 110 |
| T T T Y G G G A A A Y G Y G A G G A | 111 |
| T T Y G G G A A A Y G Y G A G G A T | 112 |
| T Y G G G A A A Y G Y G A G G A T G | 113 |
| Y G G G A A A Y G Y G A G G A T G T | 114 |
| G G G A A A Y G Y G A G G A T G T T | 115 |
| G G A A A Y G Y G A G G A T G T T T | 116 |
| G A A A Y G Y G A G G A T G T T T T | 117 |
| A A A Y G Y G A G G A T G T T T T A | 118 |
| A A Y G Y G A G G A T G T T T T A T | 119 |
| A Y G Y G A G G A T G T T T T A T G | 120 |
| Y G Y G A G G A T G T T T T A T G G | 121 |
| G Y G A G G A T G T T T T A T G G A | 122 |
| Y G A G G A T G T T T T A T G G A G | 123 |
| G A G G A T G T T T T A T G G A G Y | 124 |
| A G G A T G T T T T A T G G A G Y G | 125 |
| G G A T G T T T T A T G G A G Y G T | 126 |
| G A T G T T T T A T G G A G Y G T G | 127 |
| A T G T T T T A T G G A G Y G T G A | 128 |
| T G T T T T A T G G A G Y G T G A G | 129 |
| G T T T T A T G G A G Y G T G A G T | 130 |
| T T T T A T G G A G Y G T G A G T A | 131 |
| T T T A T G G A G Y G T G A G T A T | 132 |
| T T A T G G A G Y G T G A G T A T T | 133 |
| T A T G G A G Y G T G A G T A T T T | 134 |
| A T G G A G Y G T G A G T A T T T A | 135 |
| T G G A G Y G T G A G T A T T T A A | 136 |
| G G A G Y G T G A G T A T T T A A T | 137 |
| G A G Y G T G A G T A T T T A A T T | 138 |
| A G Y G T G A G T A T T T A A T T T | 139 |
| G Y G T G A G T A T T T A A T T T T | 140 |
| Y G T G A G T A T T T A A T T T T T | 141 |
| G T G A G T A T T T A A T T T T T T | 142 |
| T G A G T A T T T A A T T T T T T T | 143 |
| G A G T A T T T A A T T T T T T T T | 144 |
| A G T A T T T A A T T T T T T T T T | 145 |
| G T A T T T A A T T T T T T T T T T | 146 |
| T A T T T A A T T T T T T T T T T A | 147 |
| A T T T A A T T T T T T T T T T A T | 148 |
| T T T A A T T T T T T T T T T A T A | 149 |
| T T A A T T T T T T T T T T A T A T | 150 |
| T A A T T T T T T T T T T A T A T A | 151 |
| A A T T T T T T T T T T A T A T A A | 152 |
| A T T T T T T T T T T A T A T A A A | 153 |
| T T T T T T T T T T A T A T A A A A | 154 |
| T T T T T T T T T A T A T A A A A T | 155 |
| T T T T T T T T A T A T A A A A T T | 156 |
| T T T T T T T A T A T A A A A T T T | 157 |
| T T T T T T A T A T A A A A T T T G | 158 |
| T T T T T A T A T A A A A T T T G T | 159 |
| T T T T A T A T A A A A T T T G T T | 160 |
| T T T A T A T A A A A T T T G T T T | 161 |
| T T A T A T A A A A T T T G T T T G | 162 |
| T A T A T A A A A T T T G T T T G T | 163 |
| A T A T A A A A T T T G T T T G T T | 164 |
| T A T A A A A T T T G T T T G T T Y | 165 |
| A T A A A A T T T G T T T G T T Y G | 166 |
| T A A A A T T T G T T T G T T Y G T | 167 |
| A A A A T T T G T T T G T T Y G T T | 168 |
| A A A T T T G T T T G T T Y G T T T | 169 |
| A A T T T G T T T G T T Y G T T T T | 170 |
| A T T T G T T T G T T Y G T T T T T | 171 |
| T T T G T T T G T T Y G T T T T T T | 172 |
| T T G T T T G T T Y G T T T T T T T | 173 |
| T G T T T G T T Y G T T T T T T T G | 174 |
| G T T T G T T Y G T T T T T T T G G | 175 |
| T T T G T T Y G T T T T T T T G G T | 176 |
| T T G T T Y G T T T T T T T G G T T | 177 |
| T G T T Y G T T T T T T T G G T T T | 178 |
| G T T Y G T T T T T T T G G T T T T | 179 |
| T T Y G T T T T T T T G G T T T T T | 180 |
| T Y G T T T T T T T G G T T T T T T | 181 |
| Y G T T T T T T T G G T T T T T T T | 182 |
| G T T T T T T T G G T T T T T T T T | 183 |
| T T T T T T T G G T T T T T T T T T | 184 |
| T T T T T T G G T T T T T T T T T G | 185 |
| T T T T T G G T T T T T T T T T G T | 186 |
| T T T T G G T T T T T T T T T G T A | 187 |
| T T T G G T T T T T T T T T G T A A | 188 |
| T T G G T T T T T T T T T G T A A A | 189 |
| T G G T T T T T T T T T G T A A A G | 190 |
| G G T T T T T T T T T G T A A A G T | 191 |
| G T T T T T T T T T G T A A A G T A | 192 |
| T T T T T T T T T G T A A A G T A A | 193 |
| T T T T T T T T G T A A A G T A A G | 194 |
| T T T T T T T G T A A A G T A A G T | 195 |
| T T T T T T G T A A A G T A A G T A | 196 |
| T T T T T G T A A A G T A A G T A A | 197 |
| T T T T G T A A A G T A A G T A A G | 198 |
| T T T G T A A A G T A A G T A A G T | 199 |
| T T G T A A A G T A A G T A A G T T | 200 |
| T G T A A A G T A A G T A A G T T G | 201 |
| G T A A A G T A A G T A A G T T G Y | 202 |
| T A A A G T A A G T A A G T T G Y G | 203 |
| A A A G T A A G T A A G T T G Y G T | 204 |
| A A G T A A G T A A G T T G Y G T T | 205 |
| A G T A A G T A A G T T G Y G T T T | 206 |
| G T A A G T A A G T T G Y G T T T G | 207 |
| T A A G T A A G T T G Y G T T T G G | 208 |
| A A G T A A G T T G Y G T T T G G T | 209 |
| A G T A A G T T G Y G T T T G G T A | 210 |
| G T A A G T T G Y G T T T G G T A A | 211 |
| T A A G T T G Y G T T T G G T A A A | 212 |
| A A G T T G Y G T T T G G T A A A T | 213 |
| A G T T G Y G T T T G G T A A A T A | 214 |
| G T T G Y G T T T G G T A A A T A A | 215 |
| T T G Y G T T T G G T A A A T A A T | 216 |
| T G Y G T T T G G T A A A T A A T G | 217 |
| G Y G T T T G G T A A A T A A T G A | 218 |
| Y G T T T G G T A A A T A A T G A A | 219 |
| G T T T G G T A A A T A A T G A A A | 220 |
| T T T G G T A A A T A A T G A A A T | 221 |
| T T G G T A A A T A A T G A A A T G | 222 |
| T G G T A A A T A A T G A A A T G G | 223 |
| G G T A A A T A A T G A A A T G G A | 224 |
| G T A A A T A A T G A A A T G G A A | 225 |
| T A A A T A A T G A A A T G G A A G | 226 |
| A A A T A A T G A A A T G G A A G T | 227 |
| A A T A A T G A A A T G G A A G T G | 228 |
| A T A A T G A A A T G G A A G T G T | 229 |
| T A A T G A A A T G G A A G T G T A | 230 |
| A A T G A A A T G G A A G T G T A A | 231 |
| A T G A A A T G G A A G T G T A A G | 232 |
| T G A A A T G G A A G T G T A A G G | 233 |
| G A A A T G G A A G T G T A A G G A | 234 |
| A A A T G G A A G T G T A A G G A G | 235 |
| A A T G G A A G T G T A A G G A G G | 236 |
| A T G G A A G T G T A A G G A G G T | 237 |
| T G G A A G T G T A A G G A G G T T | 238 |
| G G A A G T G T A A G G A G G T T A | 239 |
| G A A G T G T A A G G A G G T T A A | 240 |
| A A G T G T A A G G A G G T T A A G | 241 |
| A G T G T A A G G A G G T T A A G T | 242 |
| G T G T A A G G A G G T T A A G T T | 243 |
| T G T A A G G A G G T T A A G T T A | 244 |
| G T A A G G A G G T T A A G T T A A | 245 |
| T A A G G A G G T T A A G T T A A T | 246 |
| A A G G A G G T T A A G T T A A T A | 247 |
| A G G A G G T T A A G T T A A T A G | 248 |
| G G A G G T T A A G T T A A T A G G | 249 |
| G A G G T T A A G T T A A T A G G T | 250 |
| A G G T T A A G T T A A T A G G T G | 251 |
| G G T T A A G T T A A T A G G T G G | 252 |
| G T T A A G T T A A T A G G T G G T | 253 |
| T T A A G T T A A T A G G T G G T A | 254 |
| T A A G T T A A T A G G T G G T A A | 255 |
| A A G T T A A T A G G T G G T A A Y | 256 |
| A G T T A A T A G G T G G T A A Y G | 257 |
| G T T A A T A G G T G G T A A Y G G | 258 |
| T T A A T A G G T G G T A A Y G G G | 259 |
| T A A T A G G T G G T A A Y G G G T | 260 |
| A A T A G G T G G T A A Y G G G T T | 261 |
| A T A G G T G G T A A Y G G G T T A | 262 |
| T A G G T G G T A A Y G G G T T A A | 263 |
| A G G T G G T A A Y G G G T T A A T | 264 |
| G G T G G T A A Y G G G T T A A T A | 265 |
| G T G G T A A Y G G G T T A A T A A | 266 |
| T G G T A A Y G G G T T A A T A A G | 267 |
| G G T A A Y G G G T T A A T A A G T | 268 |
| G T A A Y G G G T T A A T A A G T G | 269 |
| T A A Y G G G T T A A T A A G T G T | 270 |
| A A Y G G G T T A A T A A G T G T T | 271 |
| A Y G G G T T A A T A A G T G T T G | 272 |
| Y G G G T T A A T A A G T G T T G G | 273 |
| G G G T T A A T A A G T G T T G G Y | 274 |
| G G T T A A T A A G T G T T G G Y G | 275 |
| G T T A A T A A G T G T T G G Y G Y | 276 |
| T T A A T A A G T G T T G G Y G Y G | 277 |
| T A A T A A G T G T T G G Y G Y G G | 278 |
| A A T A A G T G T T G G Y G Y G G G | 279 |
| A T A A G T G T T G G Y G Y G G G G | 280 |
| T A A G T G T T G G Y G Y G G G G T | 281 |
| A A G T G T T G G Y G Y G G G G T T | 282 |
| A G T G T T G G Y G Y G G G G T T Y | 283 |
| G T G T T G G Y G Y G G G G T T Y G | 284 |
| T G T T G G Y G Y G G G G T T Y G T | 285 |
| G T T G G Y G Y G G G G T T Y G T T | 286 |
| T T G G Y G Y G G G G T T Y G T T A | 287 |
| T G G Y G Y G G G G T T Y G T T A G | 288 |
| G G Y G Y G G G G T T Y G T T A G G | 289 |
| G Y G Y G G G G T T Y G T T A G G G | 290 |
| Y G Y G G G G T T Y G T T A G G G T | 291 |
| G Y G G G G T T Y G T T A G G G T G | 292 |
| Y G G G G T T Y G T T A G G G T G G | 293 |
| G G G G T T Y G T T A G G G T G G A | 294 |
| G G G T T Y G T T A G G G T G G A G | 295 |
| G G T T Y G T T A G G G T G G A G G | 296 |
| G T T Y G T T A G G G T G G A G G T | 297 |
| T T Y G T T A G G G T G G A G G T T | 298 |
| T Y G T T A G G G T G G A G G T T G | 299 |
| T Y G T T A G G G T G G A G G T T G | 300 |
| Y G T T A G G G T G G A G G T T G A | 301 |
| G T T A G G G T G G A G G T T G A G | 302 |
| T T A G G G T G G A G G T T G A G A | 303 |
| T A G G G T G G A G G T T G A G A A | 304 |
| A G G G T G G A G G T T G A G A A Y | 305 |
| G G G T G G A G G T T G A G A A Y G | 306 |
| G G T G G A G G T T G A G A A Y G T | 307 |
| G T G G A G G T T G A G A A Y G T T | 308 |
| T G G A G G T T G A G A A Y G T T T | 309 |
| G G A G G T T G A G A A Y G T T T T | 310 |
| G A G G T T G A G A A Y G T T T T T | 311 |
| A G G T T G A G A A Y G T T T T T T | 312 |
| G G T T G A G A A Y G T T T T T T Y | 313 |
| G T T G A G A A Y G T T T T T T Y G | 314 |
| T T G A G A A Y G T T T T T T Y G G | 315 |
| T G A G A A Y G T T T T T T Y G G G | 316 |
| G A G A A Y G T T T T T T Y G G G T | 317 |
| A G A A Y G T T T T T T Y G G G T G | 318 |
| G A A Y G T T T T T T Y G G G T G G | 319 |
| A A Y G T T T T T T Y G G G T G G T | 320 |
| A Y G T T T T T T Y G G G T G G T T | 321 |
| Y G T T T T T T Y G G G T G G T T G | 322 |
| G T T T T T T Y G G G T G G T T G G | 323 |
| T T T T T T Y G G G T G G T T G G Y | 324 |
| T T T T T Y G G G T G G T T G G Y G | 325 |
| T T T T Y G G G T G G T T G G Y G Y | 326 |
| T T T Y G G G T G G T T G G Y G Y G | 327 |
| T T Y G G G T G G T T G G Y G Y G G | 328 |
| T Y G G G T G G T T G G Y G Y G G G | 329 |
| Y G G G T G G T T G G Y G Y G G G G | 330 |
| G G G T G G T T G G Y G Y G G G G T | 331 |
| G G T G G T T G G Y G Y G G G G T T | 332 |
| G T G G T T G G Y G Y G G G G T T G | 333 |
| T G G T T G G Y G Y G G G G T T G G | 334 |
| G G T T G G Y G Y G G G G T T G G A | 335 |
| G T T G G Y G Y G G G G T T G G A G | 336 |
| T T G G Y G Y G G G G T T G G A G A | 337 |
| T G G Y G Y G G G G T T G G A G A Y | 338 |
| G G Y G Y G G G G T T G G A G A Y G | 339 |
| G Y G Y G G G G T T G G A G A Y G G | 340 |
| Y G Y G G G G T T G G A G A Y G G T | 341 |
| G Y G G G G T T G G A G A Y G G T T | 342 |
| Y G G G G T T G G A G A Y G G T T Y | 343 |
| G G G G T T G G A G A Y G G T T Y G | 344 |
| G G G T T G G A G A Y G G T T Y G Y | 345 |
| G G T T G G A G A Y G G T T Y G Y G | 346 |
| G T T G G A G A Y G G T T Y G Y G A | 347 |
| T T G G A G A Y G G T T Y G Y G A G | 348 |
| T G G A G A Y G G T T Y G Y G A G T | 349 |
| G G A G A Y G G T T Y G Y G A G T G | 350 |
| G A G A Y G G T T Y G Y G A G T G T | 351 |
| A G A Y G G T T Y G Y G A G T G T G | 352 |
| G A Y G G T T Y G Y G A G T G T G A | 353 |
| A Y G G T T Y G Y G A G T G T G A G | 354 |
| Y G G T T Y G Y G A G T G T G A G Y | 355 |
| G G T T Y G Y G A G T G T G A G Y G | 356 |
| G T T Y G Y G A G T G T G A G Y G G | 357 |
| T T Y G Y G A G T G T G A G Y G G Y | 358 |
| T Y G Y G A G T G T G A G Y G G Y G | 359 |
| Y G Y G A G T G T G A G Y G G Y G T | 360 |
| G Y G A G T G T G A G Y G G Y G T T | 361 |
| Y G A G T G T G A G Y G G Y G T T T | 362 |
| G A G T G T G A G Y G G Y G T T T G | 363 |
| A G T G T G A G Y G G Y G T T T G T | 364 |
| G T G T G A G Y G G Y G T T T G T T | 365 |
| T G T G A G Y G G Y G T T T G T T T | 366 |
| G T G A G Y G G Y G T T T G T T T A | 367 |
| T G A G Y G G Y G T T T G T T T A G | 368 |
| G A G Y G G Y G T T T G T T T A G G | 369 |
| A G Y G G Y G T T T G T T T A G G G | 370 |
| G Y G G Y G T T T G T T T A G G G T | 371 |
| Y G G Y G T T T G T T T A G G G T A | 372 |
| G G Y G T T T G T T T A G G G T A G | 373 |
| G Y G T T T G T T T A G G G T A G A | 374 |
| Y G T T T G T T T A G G G T A G A T | 375 |
| G T T T G T T T A G G G T A G A T A | 376 |
| T T T G T T T A G G G T A G A T A G | 377 |
| T T G T T T A G G G T A G A T A G T | 378 |
| T G T T T A G G G T A G A T A G T T | 379 |
| G T T T A G G G T A G A T A G T T G | 380 |
| T T T A G G G T A G A T A G T T G A | 381 |
| T T A G G G T A G A T A G T T G A G | 382 |
| T A G G G T A G A T A G T T G A G G | 383 |
| A G G G T A G A T A G T T G A G G G | 384 |
| G G G T A G A T A G T T G A G G G Y | 385 |
| G G T A G A T A G T T G A G G G Y G | 386 |
| G T A G A T A G T T G A G G G Y G G | 387 |
| T A G A T A G T T G A G G G Y G G G | 388 |
| A G A T A G T T G A G G G Y G G G G | 389 |
| G A T A G T T G A G G G Y G G G G G | 390 |
| A T A G T T G A G G G Y G G G G G T | 391 |
| T A G T T G A G G G Y G G G G G T G | 392 |
| A G T T G A G G G Y G G G G G T G G | 393 |
| G T T G A G G G Y G G G G G T G G A | 394 |
| T T G A G G G Y G G G G G T G G A T | 395 |
| T G A G G G Y G G G G G T G G A T G | 396 |
| G A G G G Y G G G G G T G G A T G T | 397 |
| A G G G Y G G G G G T G G A T G T T | 398 |
| G G G Y G G G G G T G G A T G T T G | 399 |
| G G Y G G G G G T G G A T G T T G G | 400 |
| G Y G G G G G T G G A T G T T G G A | 401 |
| Y G G G G G T G G A T G T T G G A T | 402 |
| G G G G G T G G A T G T T G G A T G | 403 |
| G G G G T G G A T G T T G G A T G G | 404 |
| G G G T G G A T G T T G G A T G G A | 405 |
| G G T G G A T G T T G G A T G G A T | 406 |
| G T G G A T G T T G G A T G G A T T | 407 |
| T G G A T G T T G G A T G G A T T A | 408 |
| G G A T G T T G G A T G G A T T A G | 409 |
| G A T G T T G G A T G G A T T A G A | 410 |
| A T G T T G G A T G G A T T A G A A | 411 |
| T G T T G G A T G G A T T A G A A T | 412 |
| G T T G G A T G G A T T A G A A T T | 413 |
| T T G G A T G G A T T A G A A T T A | 414 |
| T G G A T G G A T T A G A A T T A T | 415 |
| G G A T G G A T T A G A A T T A T T | 416 |
| G A T G G A T T A G A A T T A T T A | 417 |
| A T G G A T T A G A A T T A T T A T | 418 |
| T G G A T T A G A A T T A T T A T A | 419 |
| G G A T T A G A A T T A T T A T A T | 420 |
| G A T T A G A A T T A T T A T A T T | 421 |
| A T T A G A A T T A T T A T A T T T | 422 |
| T T A G A A T T A T T A T A T T T G | 423 |
| T A G A A T T A T T A T A T T T G G | 424 |
| A G A A T T A T T A T A T T T G G G | 425 |
| G A A T T A T T A T A T T T G G G T | 426 |
| A A T T A T T A T A T T T G G G T T | 427 |
| A T T A T T A T A T T T G G G T T T | 428 |
| T T A T T A T A T T T G G G T T T G | 429 |
| T A T T A T A T T T G G G T T T G T | 430 |
| A T T A T A T T T G G G T T T G T T | 431 |
| T T A T A T T T G G G T T T G T T G | 432 |
| T A T A T T T G G G T T T G T T G T | 433 |
| A T A T T T G G G T T T G T T G T T | 434 |
| T A T T T G G G T T T G T T G T T T | 435 |
| A T T T G G G T T T G T T G T T T G | 436 |
| T T T G G G T T T G T T G T T T G T | 437 |
| T T G G G T T T G T T G T T T G T T | 438 |
| T G G G T T T G T T G T T T G T T T | 439 |
| G G G T T T G T T G T T T G T T T G | 440 |
| G G T T T G T T G T T T G T T T G A | 441 |
| G T T T G T T G T T T G T T T G A G | 442 |
| T T T G T T G T T T G T T T G A G T | 443 |
| T T G T T G T T T G T T T G A G T T | 444 |
| T G T T G T T T G T T T G A G T T T | 445 |
| G T T G T T T G T T T G A G T T T G | 446 |
| T T G T T T G T T T G A G T T T G A | 447 |
| T G T T T G T T T G A G T T T G A A | 448 |
| G T T T G T T T G A G T T T G A A T | 449 |
| T T T G T T T G A G T T T G A A T T | 450 |
| T T G T T T G A G T T T G A A T T A | 451 |

TABLE 2
Examples of sequence identical oligonucleotide
sequences specific for bisulfite converted methy-
lated and/or non-methylated sense strand of the
SNCA_{(-926/-483)} region of the human SNCA gene.

| | SEQ ID |
|---|---|
| Sequence | NO: |
| T A A T T C A A A C T C A A A C A A | 452 |
| A A T T C A A A C T C A A A C A A A | 453 |
| A T T C A A A C T C A A A C A A A C | 454 |
| T T C A A A C T C A A A C A A A C A | 455 |
| T C A A A C T C A A A C A A A C A A | 456 |
| C A A A C T C A A A C A A A C A A C | 457 |
| A A A C T C A A A C A A A C A A C A | 458 |
| A A C T C A A A C A A A C A A C A A | 459 |
| A C T C A A A C A A A C A A C A A A | 460 |
| C T C A A A C A A A C A A C A A A C | 461 |
| T C A A A C A A A C A A C A A A C C | 462 |
| C A A A C A A A C A A C A A A C C C | 463 |
| A A A C A A A C A A C A A A C C C A | 464 |
| A A C A A A C A A C A A A C C C A A | 465 |
| A C A A A C A A C A A A C C C A A A | 466 |
| C A A A C A A C A A A C C C A A A T | 467 |
| A A A C A A C A A A C C C A A A T A | 468 |
| A A C A A C A A A C C C A A A T A T | 469 |
| A C A A C A A A C C C A A A T A T A | 470 |
| C A A C A A A C C C A A A T A T A A | 471 |
| A A C A A A C C C A A A T A T A A T | 472 |
| A C A A A C C C A A A T A T A A T A | 473 |
| C A A A C C C A A A T A T A A T A A | 474 |
| A A A C C C A A A T A T A A T A A T | 475 |
| A A C C C A A A T A T A A T A A T T | 476 |
| A C C C A A A T A T A A T A A T T C | 477 |
| C C C A A A T A T A A T A A T T C T | 478 |
| C C A A A T A T A A T A A T T C T A | 479 |
| C A A A T A T A A T A A T T C T A A | 480 |
| A A A T A T A A T A A T T C T A A T | 481 |
| A A T A T A A T A A T T C T A A T C | 482 |
| A T A T A A T A A T T C T A A T C C | 483 |
| T A T A A T A A T T C T A A T C C A | 484 |
| A T A A T A A T T C T A A T C C A T | 485 |
| T A A T A A T T C T A A T C C A T C | 486 |
| A A T A A T T C T A A T C C A T C C | 487 |
| A T A A T T C T A A T C C A T C C A | 488 |
| T A A T T C T A A T C C A T C C A A | 489 |
| A A T T C T A A T C C A T C C A A C | 490 |
| A T T C T A A T C C A T C C A A C A | 491 |
| T T C T A A T C C A T C C A A C A T | 492 |
| T C T A A T C C A T C C A A C A T C | 493 |
| C T A A T C C A T C C A A C A T C C | 494 |
| T A A T C C A T C C A A C A T C C A | 495 |
| A A T C C A T C C A A C A T C C A C | 496 |
| A T C C A T C C A A C A T C C A C C | 497 |
| T C C A T C C A A C A T C C A C C C | 498 |
| C C A T C C A A C A T C C A C C C C | 499 |
| C A T C C A A C A T C C A C C C C C | 500 |
| A T C C A A C A T C C A C C C C C R | 501 |
| T C C A A C A T C C A C C C C C R C | 502 |
| C C A A C A T C C A C C C C C R C C | 503 |
| C A A C A T C C A C C C C C R C C C | 504 |
| A A C A T C C A C C C C C R C C C T | 505 |
| A C A T C C A C C C C C R C C C T C | 506 |
| C A T C C A C C C C C R C C C T C A | 507 |
| A T C C A C C C C C R C C C T C A A | 508 |
| T C C A C C C C C R C C C T C A A C | 509 |
| C C A C C C C C R C C C T C A A C T | 510 |
| C A C C C C C R C C C T C A A C T A | 511 |
| A C C C C C R C C C T C A A C T A T | 512 |
| C C C C C R C C C T C A A C T A T C | 513 |
| C C C C R C C C T C A A C T A T C T | 514 |
| C C C R C C C T C A A C T A T C T A | 515 |
| C C R C C C T C A A C T A T C T A C | 516 |
| C R C C C T C A A C T A T C T A C C | 517 |
| R C C C T C A A C T A T C T A C C C | 518 |
| C C C T C A A C T A T C T A C C C T | 519 |
| C C T C A A C T A T C T A C C C T A | 520 |
| C T C A A C T A T C T A C C C T A A | 521 |
| T C A A C T A T C T A C C C T A A A | 522 |
| C A A C T A T C T A C C C T A A A C | 523 |
| A A C T A T C T A C C C T A A A C A | 524 |
| A C T A T C T A C C C T A A A C A A | 525 |
| C T A T C T A C C C T A A A C A A A | 526 |
| T A T C T A C C C T A A A C A A A C | 527 |
| A T C T A C C C T A A A C A A A C R | 528 |
| T C T A C C C T A A A C A A A C R C | 529 |
| C T A C C C T A A A C A A A C R C C | 530 |
| T A C C C T A A A C A A A C R C C R | 531 |
| A C C C T A A A C A A A C R C C R C | 532 |
| C C C T A A A C A A A C R C C R C T | 533 |
| C C T A A A C A A A C R C C R C T C | 534 |
| C T A A A C A A A C R C C R C T C A | 535 |
| T A A A C A A A C R C C R C T C A C | 536 |
| A A A C A A A C R C C R C T C A C A | 537 |
| A A C A A A C R C C R C T C A C A C | 538 |
| A C A A A C R C C R C T C A C A C T | 539 |
| C A A A C R C C R C T C A C A C T C | 540 |
| A A A C R C C R C T C A C A C T C R | 541 |
| A A C R C C R C T C A C A C T C R C | 542 |
| A C R C C R C T C A C A C T C R C R | 543 |
| C R C C R C T C A C A C T C R C R A | 544 |
| R C C R C T C A C A C T C R C R A A | 545 |
| C C R C T C A C A C T C R C R A A C | 546 |
| C R C T C A C A C T C R C R A A C C | 547 |
| R C T C A C A C T C R C R A A C C R | 548 |
| C T C A C A C T C R C R A A C C R T | 549 |
| T C A C A C T C R C R A A C C R T C | 550 |
| C A C A C T C R C R A A C C R T C T | 551 |
| A C A C T C R C R A A C C R T C T C | 552 |
| C A C T C R C R A A C C R T C T C C | 553 |
| A C T C R C R A A C C R T C T C C A | 554 |
| C T C R C R A A C C R T C T C C A A | 555 |
| T C R C R A A C C R T C T C C A A C | 556 |
| C R C R A A C C R T C T C C A A C C | 557 |
| R C R A A C C R T C T C C A A C C C | 558 |
| C R A A C C R T C T C C A A C C C C | 559 |
| R A A C C R T C T C C A A C C C C R | 560 |
| A A C C R T C T C C A A C C C C R C | 561 |
| A C C R T C T C C A A C C C C R C R | 562 |
| C C R T C T C C A A C C C C R C R C | 563 |
| C R T C T C C A A C C C C R C R C C | 564 |
| R T C T C C A A C C C C R C R C C A | 565 |
| T C T C C A A C C C C R C R C C A A | 566 |
| C T C C A A C C C C R C R C C A A C | 567 |
| T C C A A C C C C R C R C C A A C C | 568 |
| C C A A C C C C R C R C C A A C C A | 569 |
| C A A C C C C R C R C C A A C C A C | 570 |
| A A C C C C R C R C C A A C C A C C | 571 |
| A C C C C R C R C C A A C C A C C C | 572 |
| C C C C R C R C C A A C C A C C C R | 573 |
| C C C R C R C C A A C C A C C C R A | 574 |
| C C R C R C C A A C C A C C C R A A | 575 |
| C R C R C C A A C C A C C C R A A A | 576 |
| R C R C C A A C C A C C C R A A A A | 577 |
| C R C C A A C C A C C C R A A A A A | 578 |
| R C C A A C C A C C C R A A A A A A | 579 |
| C C A A C C A C C C R A A A A A A C | 580 |
| C A A C C A C C C R A A A A A A C R | 581 |
| A A C C A C C C R A A A A A A C R T | 582 |
| A C C A C C C R A A A A A A C R T T | 583 |
| C C A C C C R A A A A A A C R T T C | 584 |
| C A C C C R A A A A A A C R T T C T | 585 |
| A C C C R A A A A A A C R T T C T C | 586 |
| C C C R A A A A A A C R T T C T C A | 587 |
| C C R A A A A A A C R T T C T C A A | 588 |
| C R A A A A A A C R T T C T C A A C | 589 |
| R A A A A A A C R T T C T C A A C C | 590 |
| A A A A A A C R T T C T C A A C C T | 591 |
| A A A A A C R T T C T C A A C C T C | 592 |
| A A A A C R T T C T C A A C C T C C | 593 |
| A A A C R T T C T C A A C C T C C A | 594 |
| A A C R T T C T C A A C C T C C A C | 595 |
| A C R T T C T C A A C C T C C A C C | 596 |
| C R T T C T C A A C C T C C A C C C | 597 |
| R T T C T C A A C C T C C A C C C T | 598 |
| T T C T C A A C C T C C A C C C T A | 599 |
| T C T C A A C C T C C A C C C T A A | 600 |
| C T C A A C C T C C A C C C T A A C | 601 |
| T C A A C C T C C A C C C T A A C R | 602 |
| C A A C C T C C A C C C T A A C R A | 603 |
| A A C C T C C A C C C T A A C R A A | 604 |
| A C C T C C A C C C T A A C R A A C | 605 |
| C C T C C A C C C T A A C R A A C C | 606 |
| C T C C A C C C T A A C R A A C C C | 607 |
| T C C A C C C T A A C R A A C C C C | 608 |
| C C A C C C T A A C R A A C C C C R | 609 |
| C A C C C T A A C R A A C C C C R C | 610 |
| A C C C T A A C R A A C C C C R C R | 611 |
| C C C T A A C R A A C C C C R C R C | 612 |
| C C T A A C R A A C C C C R C R C C | 613 |
| C T A A C R A A C C C C R C R C C A | 614 |
| T A A C R A A C C C C R C R C C A A | 615 |
| A A C R A A C C C C R C R C C A A C | 616 |
| A C R A A C C C C R C R C C A A C A | 617 |
| C R A A C C C C R C R C C A A C A C | 618 |
| R A A C C C C R C R C C A A C A C T | 619 |
| A A C C C C R C R C C A A C A C T T | 620 |
| A C C C C R C R C C A A C A C T T A | 621 |
| C C C C R C R C C A A C A C T T A T | 622 |
| C C C R C R C C A A C A C T T A T T | 623 |
| C C R C R C C A A C A C T T A T T A | 624 |
| C R C R C C A A C A C T T A T T A A | 625 |
| R C R C C A A C A C T T A T T A A C | 626 |
| C R C C A A C A C T T A T T A A C C | 627 |
| R C C A A C A C T T A T T A A C C C | 628 |
| C C A A C A C T T A T T A A C C C R | 629 |
| C A A C A C T T A T T A A C C C R T | 630 |
| A A C A C T T A T T A A C C C R T T | 631 |
| A C A C T T A T T A A C C C R T T A | 632 |
| C A C T T A T T A A C C C R T T A C | 633 |
| A C T T A T T A A C C C R T T A C C | 634 |
| C T T A T T A A C C C R T T A C C A | 635 |
| T T A T T A A C C C R T T A C C A C | 636 |
| T A T T A A C C C R T T A C C A C C | 637 |
| A T T A A C C C R T T A C C A C C T | 638 |
| T T A A C C C R T T A C C A C C T A | 639 |
| T A A C C C R T T A C C A C C T A T | 640 |
| A A C C C R T T A C C A C C T A T T | 641 |
| A C C C R T T A C C A C C T A T T A | 642 |
| C C C R T T A C C A C C T A T T A A | 643 |
| C C R T T A C C A C C T A T T A A C | 644 |
| C R T T A C C A C C T A T T A A C T | 645 |
| R T T A C C A C C T A T T A A C T T | 646 |
| T T A C C A C C T A T T A A C T T A | 647 |
| T A C C A C C T A T T A A C T T A A | 648 |
| A C C A C C T A T T A A C T T A A C | 649 |
| C C A C C T A T T A A C T T A A C C | 650 |
| C A C C T A T T A A C T T A A C C T | 651 |
| A C C T A T T A A C T T A A C C T C | 652 |
| C C T A T T A A C T T A A C C T C C | 653 |
| C T A T T A A C T T A A C C T C C T | 654 |
| T A T T A A C T T A A C C T C C T T | 655 |
| A T T A A C T T A A C C T C C T T A | 656 |
| T T A A C T T A A C C T C C T T A C | 657 |
| T A A C T T A A C C T C C T T A C A | 658 |
| A A C T T A A C C T C C T T A C A C | 659 |
| A C T T A A C C T C C T T A C A C T | 660 |
| C T T A A C C T C C T T A C A C T T | 661 |
| T T A A C C T C C T T A C A C T T C | 662 |
| T A A C C T C C T T A C A C T T C C | 663 |
| A A C C T C C T T A C A C T T C C A | 664 |
| A C C T C C T T A C A C T T C C A T | 665 |
| C C T C C T T A C A C T T C C A T T | 666 |
| C T C C T T A C A C T T C C A T T T | 667 |
| T C C T T A C A C T T C C A T T T C | 668 |
| C C T T A C A C T T C C A T T T C A | 669 |
| C T T A C A C T T C C A T T T C A T | 670 |
| T T A C A C T T C C A T T T C A T T | 671 |
| T A C A C T T C C A T T T C A T T A | 672 |
| A C A C T T C C A T T T C A T T A T | 673 |
| C A C T T C C A T T T C A T T A T T | 674 |
| A C T T C C A T T T C A T T A T T T | 675 |
| C T T C C A T T T C A T T A T T T A | 676 |
| T T C C A T T T C A T T A T T T A C | 677 |
| T C C A T T T C A T T A T T T A C C | 678 |
| C C A T T T C A T T A T T T A C C A | 679 |
| C A T T T C A T T A T T T A C C A A | 680 |
| A T T T C A T T A T T T A C C A A A | 681 |
| T T T C A T T A T T T A C C A A A C | 682 |
| T T C A T T A T T T A C C A A A C R | 683 |
| T C A T T A T T T A C C A A A C R C | 684 |
| C A T T A T T T A C C A A A C R C A | 685 |
| A T T A T T T A C C A A A C R C A A | 686 |
| T T A T T T A C C A A A C R C A A C | 687 |
| T A T T T A C C A A A C R C A A C T | 688 |
| A T T T A C C A A A C R C A A C T T | 689 |
| T T T A C C A A A C R C A A C T T A | 690 |
| T T A C C A A A C R C A A C T T A C | 691 |
| T A C C A A A C R C A A C T T A C T | 692 |
| A C C A A A C R C A A C T T A C T T | 693 |
| C C A A A C R C A A C T T A C T T A | 694 |
| C A A A C R C A A C T T A C T T A C | 695 |
| A A A C R C A A C T T A C T T A C T | 696 |
| A A C R C A A C T T A C T T A C T T | 697 |
| A C R C A A C T T A C T T A C T T T | 698 |
| C R C A A C T T A C T T A C T T T A | 699 |
| R C A A C T T A C T T A C T T T A C | 700 |
| C A A C T T A C T T A C T T T A C A | 701 |
| A A C T T A C T T A C T T T A C A A | 702 |
| A C T T A C T T A C T T T A C A A A | 703 |
| C T T A C T T A C T T T A C A A A A | 704 |
| T T A C T T A C T T T A C A A A A A | 705 |
| T A C T T A C T T T A C A A A A A A | 706 |
| A C T T A C T T T A C A A A A A A A | 707 |
| C T T A C T T T A C A A A A A A A A | 708 |
| T T A C T T T A C A A A A A A A A A | 709 |
| T A C T T T A C A A A A A A A A A C | 710 |
| A C T T T A C A A A A A A A A A C C | 711 |
| C T T T A C A A A A A A A A A C C A | 712 |
| T T T A C A A A A A A A A A C C A A | 713 |
| T T A C A A A A A A A A A C C A A A | 714 |
| T A C A A A A A A A A A C C A A A A | 715 |
| A C A A A A A A A A A C C A A A A A | 716 |
| C A A A A A A A A A C C A A A A A A | 717 |
| A A A A A A A A A C C A A A A A A A | 718 |
| A A A A A A A A C C A A A A A A A C | 719 |
| A A A A A A A C C A A A A A A A C R | 720 |
| A A A A A A C C A A A A A A A C R A | 721 |
| A A A A A C C A A A A A A A C R A A | 722 |
| A A A A C C A A A A A A A C R A A C | 723 |
| A A A C C A A A A A A A C R A A C A | 724 |
| A A C C A A A A A A A C R A A C A A | 725 |
| A C C A A A A A A A C R A A C A A A | 726 |
| C C A A A A A A A C R A A C A A A C | 727 |
| C A A A A A A A C R A A C A A A C A | 728 |
| A A A A A A A C R A A C A A A C A A | 729 |
| A A A A A A C R A A C A A A C A A A | 730 |
| A A A A A C R A A C A A A C A A A T | 731 |
| A A A A C R A A C A A A C A A A T T | 732 |
| A A A C R A A C A A A C A A A T T T | 733 |
| A A C R A A C A A A C A A A T T T T | 734 |
| A C R A A C A A A C A A A T T T T A | 735 |
| C R A A C A A A C A A A T T T T A T | 736 |
| R A A C A A A C A A A T T T T A T A | 737 |
| A A C A A A C A A A T T T T A T A T | 738 |
| A C A A A C A A A T T T T A T A T A | 739 |
| C A A A C A A A T T T T A T A T A A | 740 |
| A A A C A A A T T T T A T A T A A A | 741 |
| A A C A A A T T T T A T A T A A A A | 742 |
| A C A A A T T T T A T A T A A A A A | 743 |
| C A A A T T T T A T A T A A A A A A | 744 |
| A A A T T T T A T A T A A A A A A A | 745 |
| A A T T T T A T A T A A A A A A A A | 746 |
| A T T T T A T A T A A A A A A A A A | 747 |
| T T T T A T A T A A A A A A A A A A | 748 |
| T T T A T A T A A A A A A A A A A T | 749 |
| T T A T A T A A A A A A A A A A T T | 750 |
| T A T A T A A A A A A A A A A T T A | 751 |
| A T A T A A A A A A A A A A T T A A | 752 |
| T A T A A A A A A A A A A T T A A A | 753 |
| A T A A A A A A A A A A T T A A A T | 754 |
| T A A A A A A A A A A T T A A A T A | 755 |
| A A A A A A A A A A T T A A A T A C | 756 |
| A A A A A A A A A T T A A A T A C T | 757 |
| A A A A A A A A T T A A A T A C T C | 758 |
| A A A A A A A T T A A A T A C T C A | 759 |
| A A A A A A T T A A A T A C T C A C | 760 |
| A A A A A T T A A A T A C T C A C R | 761 |
| A A A A T T A A A T A C T C A C R C | 762 |
| A A A T T A A A T A C T C A C R C T | 763 |
| A A T T A A A T A C T C A C R C T C | 764 |
| A T T A A A T A C T C A C R C T C C | 765 |
| T T A A A T A C T C A C R C T C C A | 766 |
| T A A A T A C T C A C R C T C C A T | 767 |
| T A A A T A C T C A C R C T C C A T | 768 |
| A A A T A C T C A C R C T C C A T A | 769 |
| A A T A C T C A C R C T C C A T A A | 770 |
| A T A C T C A C R C T C C A T A A A | 771 |
| T A C T C A C R C T C C A T A A A A | 772 |
| A C T C A C R C T C C A T A A A A C | 773 |
| C T C A C R C T C C A T A A A A C A | 774 |
| T C A C R C T C C A T A A A A C A T | 775 |
| C A C R C T C C A T A A A A C A T C | 776 |
| A C R C T C C A T A A A A C A T C C | 777 |
| C R C T C C A T A A A A C A T C C T | 778 |
| R C T C C A T A A A A C A T C C T C | 779 |
| C T C C A T A A A A C A T C C T C R | 780 |
| T C C A T A A A A C A T C C T C R C | 781 |
| C C A T A A A A C A T C C T C R C R | 782 |
| C A T A A A A C A T C C T C R C R T | 783 |
| A T A A A A C A T C C T C R C R T T | 784 |
| T A A A A C A T C C T C R C R T T T | 785 |
| A A A A C A T C C T C R C R T T T C | 786 |
| A A A C A T C C T C R C R T T T C C | 787 |
| A A C A T C C T C R C R T T T C C C | 788 |
| A C A T C C T C R C R T T T C C C R | 789 |
| C A T C C T C R C R T T T C C C R A | 790 |
| A T C C T C R C R T T T C C C R A A | 791 |
| T C C T C R C R T T T C C C R A A A | 792 |
| C C T C R C R T T T C C C R A A A A | 793 |
| C T C R C R T T T C C C R A A A A A | 794 |
| T C R C R T T T C C C R A A A A A A | 795 |
| C R C R T T T C C C R A A A A A A A | 796 |
| R C R T T T C C C R A A A A A A A A | 797 |
| C R T T T C C C R A A A A A A A A C | 798 |
| R T T T C C C R A A A A A A A A C R | 799 |
| T T T C C C R A A A A A A A A C R A | 800 |
| T T C C C R A A A A A A A A C R A A | 801 |
| T C C C R A A A A A A A A C R A A T | 802 |
| C C C R A A A A A A A A C R A A T C | 803 |
| C C R A A A A A A A A C R A A T C C | 804 |
| C R A A A A A A A A C R A A T C C C | 805 |
| R A A A A A A A A C R A A T C C C R | 806 |
| A A A A A A A A C R A A T C C C R A | 807 |
| A A A A A A A C R A A T C C C R A A | 808 |
| A A A A A A C R A A T C C C R A A A | 809 |
| A A A A A C R A A T C C C R A A A A | 810 |
| A A A A C R A A T C C C R A A A A A | 811 |
| A A A C R A A T C C C R A A A A A A | 812 |
| A A C R A A T C C C R A A A A A A C | 813 |
| A C R A A T C C C R A A A A A A C A | 814 |
| C R A A T C C C R A A A A A A C A A | 815 |
| R A A T C C C R A A A A A A C A A C | 816 |
| A A T C C C R A A A A A A C A A C C | 817 |
| A T C C C R A A A A A A C A A C C T | 818 |
| T C C C R A A A A A A C A A C C T A | 819 |
| C C C R A A A A A A C A A C C T A A | 820 |
| C C R A A A A A A C A A C C T A A T | 821 |
| C R A A A A A A C A A C C T A A T C | 822 |
| R A A A A A A C A A C C T A A T C T | 823 |
| A A A A A A C A A C C T A A T C T C | 824 |
| A A A A A C A A C C T A A T C T C T | 825 |
| A A A A C A A C C T A A T C T C T C | 826 |
| A A A C A A C C T A A T C T C T C A | 827 |
| A A C A A C C T A A T C T C T C A A | 828 |
| A C A A C C T A A T C T C T C A A C | 829 |
| C A A C C T A A T C T C T C A A C C | 830 |
| A A C C T A A T C T C T C A A C C C | 831 |
| A C C T A A T C T C T C A A C C C T | 832 |
| C C T A A T C T C T C A A C C C T T | 833 |
| C T A A T C T C T C A A C C C T T A | 834 |
| T A A T C T C T C A A C C C T T A T | 835 |
| A A T C T C T C A A C C C T T A T A | 836 |
| A T C T C T C A A C C C T T A T A A | 837 |
| T C T C T C A A C C C T T A T A A A | 838 |
| C T C T C A A C C C T T A T A A A A | 839 |
| T C T C A A C C C T T A T A A A A A | 840 |
| C T C A A C C C T T A T A A A A A A | 841 |
| T C A A C C C T T A T A A A A A A A | 842 |
| C A A C C C T T A T A A A A A A A A | 843 |
| A A C C C T T A T A A A A A A A A A | 844 |
| A C C C T T A T A A A A A A A A A A | 845 |
| C C C T T A T A A A A A A A A A A A | 846 |
| C C T T A T A A A A A A A A A A A T | 847 |
| C T T A T A A A A A A A A A A A T A | 848 |
| T T A T A A A A A A A A A A A T A T | 849 |
| T A T A A A A A A A A A A A T A T C | 850 |
| A T A A A A A A A A A A A T A T C A | 851 |
| T A A A A A A A A A A A T A T C A A | 852 |
| A A A A A A A A A A A T A T C A A A | 853 |
| A A A A A A A A A A T A T C A A A A | 854 |
| A A A A A A A A A T A T C A A A A A | 855 |
| A A A A A A A A T A T C A A A A A C | 856 |
| A A A A A A A T A T C A A A A A C A | 857 |
| A A A A A A T A T C A A A A A C A A | 858 |
| A A A A A T A T C A A A A A C A A A | 859 |
| A A A A T A T C A A A A A C A A A A | 860 |
| A A A T A T C A A A A A C A A A A C | 861 |
| A A T A T C A A A A A C A A A A C R | 862 |
| A T A T C A A A A A C A A A A C R A | 863 |
| T A T C A A A A A C A A A A C R A A | 864 |
| A T C A A A A A C A A A A C R A A A | 865 |
| T C A A A A A C A A A A C R A A A A | 866 |
| C A A A A A C A A A A C R A A A A C | 867 |
| A A A A A C A A A A C R A A A A C C | 868 |
| A A A A C A A A A C R A A A A C C A | 869 |
| A A A C A A A A C R A A A A C C A A | 870 |
| A A C A A A A C R A A A A C C A A A | 871 |
| A C A A A A C R A A A A C C A A A T | 872 |
| C A A A A C R A A A A C C A A A T C | 873 |
| A A A A C R A A A A C C A A A T C A | 874 |
| A A A C R A A A A C C A A A T C A A | 875 |
| A A C R A A A A C C A A A T C A A A | 876 |
| A C R A A A A C C A A A T C A A A T | 877 |
| C R A A A A C C A A A T C A A A T C | 878 |
| R A A A A C C A A A T C A A A T C T | 879 |
| A A A A C C A A A T C A A A T C T C | 880 |
| A A A C C A A A T C A A A T C T C T | 881 |
| A A C C A A A T C A A A T C T C T T | 882 |
| A C C A A A T C A A A T C T C T T T | 883 |
| C C A A A T C A A A T C T C T T T C | 884 |
| C A A A T C A A A T C T C T T T C C | 885 |
| A A A T C A A A T C T C T T T C C T | 886 |
| A A T C A A A T C T C T T T C C T T | 887 |
| A T C A A A T C T C T T T C C T T A | 888 |
| T C A A A T C T C T T T C C T T A A | 889 |
| C A A A T C T C T T T C C T T A A A | 890 |
| A A A T C T C T T T C C T T A A A C | 891 |
| A A T C T C T T T C C T T A A A C T | 892 |
| A T C T C T T T C C T T A A A C T C | 893 |
| T C T C T T T C C T T A A A C T C C | 894 |
| C T C T T T C C T T A A A C T C C C | 895 |
| T C T T T C C T T A A A C T C C C C | 896 |
| C T T T C C T T A A A C T C C C C A | 897 |
| T T T C C T T A A A C T C C C C A A | 898 |

Table 3: Examples of complementary oligonucleotide sequences specific for bisulfite converted methylated and/or non-methylated antisense strand of the SNCA_{(-926,-483)} region of the human SNCA gene.

Within the nucleotide sequences set forth in Table 1, “Y” represents C or T, wherein Y=C is specific for a methylated cytosine in the non-bisulfite converted DNA of the SNCA_{(-926,-483)} region of the human SNCA gene, and Y=T is specific for a non-methylated cytosine of a CpG dinucleotide in the non-bisulfite converted DNA of the SNCA_{(-926,-483)} region of the human SNCA gene.

Within the nucleotide sequences set forth in Table 2, “R” represents G or A, wherein R=G is specific for a methylated cytosine in the non-bisulfite converted DNA of the SNCA_{(-926/-483)} region of the human SNCA gene, and R=A is specific for a non-methylated cytosine of a CpG dinucleotide in the non-bisulfite converted DNA of the SNCA_{(-926,-483)} region of the human SNCA gene.

For amplification reactions of the SNCA_{(-926,-483)} region of the human SNCA gene or at least of portions of SNCA_{(-926,-483)} region of the human SNCA gene comprising at least one of the 23 CpG dinucleotides present in the SNCA_{(-926,-483)} region of the human SNCA gene, the forward oligonucleotide primer can, for example, be selected from the group of oligonucleotides comprising or consisting of the nucleotide sequences selected from the group consisting of SEQ ID NO. 5 to SEQ ID NO: 451. For amplification reactions of the SNCA_{(-926/-483)} region of the human SNCA gene or at least of portions of SNCA_{(-926/-483)} region of the human SNCA gene comprising at least one of the 23 CpG dinucleotides present in the SNCA_{(-926/-483)} region of the human SNCA gene, the reverse oligonucleotide primer can, for example, be selected from the group of oligonucleotides comprising or consisting of the nucleotide sequences selected from the group consisting of SEQ ID NO. 452 to SEQ ID NO: 898.

A primer for sequence analysis of the cloned PCR products from bisulfite converted DNA from the SNCA_{(-926,-483)} region of the human SNCA gene can, for example, be selected from the group of oligonucleotides comprising or consisting of the nucleotide sequences selected from the group consisting of SEQ ID NO. 5 to SEQ ID NO: 451 and SEQ ID NO. 452 to SEQ ID NO: 898.

In an embodiment of the present invention, methylation of the human SNCA-gene, of intron 1 of the human SNCA gene, and/or of the SNCA_{(-926,-483)} region of the human SNCA gene, in particular of one or more CpG dinucleotides of the SNCA_{(-926,-483)} region of the human SNCA gene, for example, of one or more CpG dinculeotides of the SNCA_{(-926,-483)} region of the human SNCA gene selected from the group consisting of CpG dinucleotides No. 1, 2, 7, 8, 11, 12, 13, 18, 21, 22 and 23 of the SNCA_{(-926,-483)} region of the human SNCA gene, and, for example, of one or more CpG dinucleotides selected from the group consisting of CpG dinucleotides No. 1, 7, 8, 13, 22 and 23 of the SNCA_{(-926,-483)} region of the human SNCA gene, is determined by analyzing restriction enzyme digestions.

Analyzing restriction enzyme digestion of DNA to analyze the methylation status of said DNA is based on the property of some restriction enzymes to be unable to cut methylated DNA. Since in eukaryotic DNA only cytosine in CG context can be methylated, restriction enzymes with CG sequences within their restriction sites come may be used. Two classical enzyme pairs are, for example, HpaII-MspI (CCGG) and SmaI-XmaI (CCCGGG). Both enzymes of each pair recognize nucleotides sequences comprising a CCGG sequence. HpaII and SmaI are, however, unable to cut DNA when the internal cytosine is methylated. This property makes HpaII-MspI and SmaI-XmaI valuable tools for rapid methylation analysis.

Prior to the methylation analysis by restriction enzyme digestion, the nucleotide sequence of the DNA fragment to be investigated has to be analyzed for the presence of CpG dinucleotides and specific restriction sites for enzyme pairs wherein one isoschizomer hydrolyses the DNA even if the cytosine of the CpG dinucleotide within the recognition sequence is methylated, and wherein the other isoschizomer does not. After restriction enzyme digestion, the DNA fragments will be separated by gel electrophoresis. A Southern blot hybridization of the separated DNA fragments has to be performed, wherein the probe for used for Southern blot hybridization should be located within the region to be analyzed and cover it completely or at least partially.

In an embodiment of the present invention, a method of determining the methylation pattern of the human SNCA gene for diagnosing and/or assessing a patient's risk of developing Parkinson's disease is provided, in particular of developing sporadic PD, wherein the SNCA gene is obtained from DNA of peripheral blood monocytes of said patient. The method according to the embodiment permits the establishment of a method for diagnosing Parkinson's disease, even if no neurological symptoms associated with Parkinson's disease are present in the patient yet. The method according to the embodiment of the present invention as well as the diagnostic assay that is based on the method according to an embodiment of the present invention are inexpensive and easy to perform without affecting the patient's acceptance.

In an embodiment of the method according to the present invention, the methylation pattern of intron 1 of the human SNCA gene from genomic DNA of the patient's peripheral blood monocytes can, for example, be determined.

In an embodiment of the method according to the present invention, the methylation pattern of the SNCA_{(-926,-483)} region of the human SNCA gene from genomic DNA of the patient's peripheral blood monocytes can, for example, be determined.

In an embodiment of the method according to the present invention, the methylation status of one or more CpG dinucleotides of the SNCA_{(-926,-483)} region of the human SNCA gene can, for example, be determined. Said one or more CpG dinucleotides can, for example, be selected from the group consisting of CpG dinucleotides No. 1, 2, 7, 8, 11, 12, 13, 18, 21, 22 and 23 of the SNCA_{(-926,-483)} region of the human SNCA gene. Said one or more CpG dinucleotides can, for example, be selected from the group consisting of CpG dinucleotides No. 1, 7, 8, 13, 22 and 23 of the SNCA_{(-926,-483)} region of the human SNCA gene.

Notwithstanding that virtually any method for analyzing the methylation status of a given CpG dinucleotide can be employed for analyzing the methylation pattern of the human SNCA gene, of intron 1 of the human SNCA gene, and/or of the SNCA_{(-926,-483)} region of the human SNCA gene, in particular of one or more CpG dinucleotides of the SNCA_{(-926,-483)} region of the human SNCA gene, one or more CpG dinculeotides of the SNCA_{(-926,-483)} region of the human SNCA gene can, for example, be selected from the group consisting of CpG dinucleotides 1, 2, 7, 8, 11, 12, 13, 18, 21, 22 and 23 of the SNCA_{(-926,-483)} region of the human SNCA gene, and, for example, said one or more CpG dinucleotides is/are selected from the group consisting of CpG dinucleotides No. 1, 7, 8, 13, 22 and 23 of the SNCA_{(-926/-483)} region of the human SNCA gene, the methylation status can, for example, be determined by one or more methods selected from the group consisting of methylation specific PCR, direct sequence analysis of bisulfite-treated DNA, COBRA-Assay and methylation-specific restriction enzyme digestion as described in more detail herein above.

In an embodiment of the method according to the present invention, methylation of the human SNCA-gene, of intron 1 of the human SNCA gene, and/or of the SNCA_{(-926,-483)} region of the human SNCA gene, in particular of one or more CpG dinucleotides of the SNCA_{(-926/-483)} region of the human SNCA gene can, for example, be determined by methylation specific PCR (MSP). Said one or more CpG dinucleotides of the SNCA_{(-926,-483)} region of the human SNCA gene can, for example, be selected from the group consisting of CpG dinucleotides 1, 2, 7, 8, 11, 12, 13, 18, 21, 22 and 23 of the SNCA_{(-926,-483)} region of the human SNCA gene, and, for example, said one or more CpG dinucleotides is/are selected from the group consisting of CpG dinucleotides No. 1, 7, 8, 13, 22 and 23 of the SNCA_{(-926/-483)} region of the human SNCA gene,

In an embodiment of the method according to the present invention, methylation of the human SNCA-gene, of intron 1 of the human SNCA gene, and/or of the SNCA_{(-926,-483)} region of the human SNCA gene, in particular of one or more CpG dinucleotides of the SNCA_{(-926/-483)} region of the human SNCA gene can, for example, be determined by sequence analysis of bisulfite treated DNA. Said one or more CpG dinucleotides of the SNCA_{(-926,-483)} region of the human SNCA gene can, for example, be selected from the group consisting of CpG dinucleotides 1, 2, 7, 8, 11, 12, 13, 18, 21, 22 and 23 of the SNCA_{(-926,-483)} region of the human SNCA gene, and, for example, said one or more CpG dinucleotides is/are selected from the group consisting of CpG dinucleotides No. 1, 7, 8, 13, 22 and 23 of the SNCA_{(-926/-483)} region of the human SNCA gene,

In an embodiment of the method according to the present invention, methylation of the human SNCA-gene, of intron 1 of the human SNCA gene, and/or of the SNCA_{(-926,-483)} region of the human SNCA gene, in particular of one or more CpG dinucleotides of the SNCA_{(-926,-483)} region of the human SNCA gene can, for example, be determined by combined bisulfite restriction analysis (COBRA assay). Said one or more CpG dinucleotides of the SNCA_{(-926/-483)} region of the human SNCA gene can, for example, be selected from the group consisting of CpG dinucleotides 1, 2, 7, 8, 11, 12, 13, 18, 21, 22 and 23 of the SNCA_{(-926,-483)} region of the human SNCA gene, and, for example, said one or more CpG dinucleotides is/are selected from the group consisting of CpG dinucleotides No. 1, 7, 8, 13, 22 and 23 of the SNCA_{(-926/-483)} region of the human SNCA gene.

In an embodiment of the method according to the present invention, methylation of the human SNCA-gene, of intron 1 of the human SNCA gene, and/or of the SNCA_{(-926,-483)} region of the human SNCA gene, in particular of one or more CpG dinucleotides of the SNCA_{(-926,-483)} region of the human SNCA gene can, for example, be determined by analyzing restriction enzyme digestions. Said one or more CpG dinucleotides of the SNCA_{(-926,-483)} region of the human SNCA gene can, for example, be selected from the group consisting of CpG dinucleotides 1, 2, 7, 8, 11, 12, 13, 18, 21, 22 and 23 of the SNCA_{(-926,-483)} region of the human SNCA gene, and, for example, said one or more CpG dinucleotides is/are selected from the group consisting of CpG dinucleotides No. 1, 7, 8, 13, 22 and 23 of the SNCA_{(-926/-483)} region of the human SNCA gene.

In an embodiment, the present invention further comprises a method for diagnosis and/or assessment of a patient's risk of developing Parkinson's disease, in particular sporadic PD, wherein said diagnostic method comprises analysis of the methylation pattern of the human SNCA gene from DNA of peripheral blood monocytes, said peripheral blood monocytes being obtained from said patient.

The method for diagnosing Parkinson's disease and/or assessing a patient's risk of developing Parkinson's disease according to an embodiment of the present invention may comprise obtaining a blood sample from said patient. Said blood sample may be specifically obtained for diagnosing Parkinson's disease or assessing the patient's risk of developing Parkinson's disease. Alternatively, blood samples of said patient may be used which were obtained from said patient for other reasons and/or earlier, wherein said blood sample had to be stored under appropriate conditions which prevent the genomic DNA from the peripheral blood monocytes of said blood sample from becoming degraded.

The method according to an embodiment of the present invention comprises isolating of peripheral blood monocytes from the patient's blood sample or blood samples. The method according to the emboiment of the present invention further comprises isolating genomic DNA from the patient's peripheral blood monocytes, and determining the methylation pattern of the patient's SNCA gene, for example, the methylation pattern of intron 1 of the patient's SNCA gene, for example, the methylation pattern of the SNCA_{(-926/-483)} region of the patient's SNCA gene. In an embodiment of the method according to the present invention, the methylation status of one or more CpG dinucleotides of the SNCA_{(-926/-483)} region of the patient's SNCA gene can, for example, be determined. The methylation status of one or more CpG dinucleotides can, for example, be determined, wherein said one or more CpG dinucleotides is/are selected from the group consisting of CpG dinucleotides No. 1, 2, 7, 8, 11, 12, 13, 18, 21, 22 and 23 of the SNCA_{(-926,-483)} region of the patient's SNCA gene is determined, and, for example, said one or more CpG dinucleotides is/are selected from the group consisting of CpG dinucleotides No. 1, 7, 8, 13, 22 and 23 of the SNCA_{(-926,-483)} region of the human SNCA gene.

Determining the methylation pattern of the patient's SNCA gene, of intron 1 of the patient's SNCA gene, and/or of the SNCA_{(-926,-483)} region of the patient's SNCA gene, more specifically the methylation status of one or more CpG dinucleotides of the SNCA_{(-926,-483)} region of the patient's SNCA gene, one or more CpG dinucleotides of the SNCA_{(-926,-483)} region of the patient's SNCA gene can, for example, be selected from the group consisting of CpG dinucleotides No. 1, 2, 7, 8, 11, 12, 13, 18, 21, 22 and 23 of the SNCA_{(-926,-483)} region of the patient's SNCA gene, and, for example, of one or more CpG dinucleotides selected from the group consisting of CpG dinucleotides No. 1, 7, 8, 13, 22 and 23 of the SNCA_{(-926,-483)} region of the patient's SNCA gene, may utilize virtually any technique that is suitable for analyzing a DNA's methylation status. However, according to an embodiment of the method according to the present invention, the methylation status is determined by one or more methods selected from the group consisting of methylation specific PCR, sequence analysis of bisulfite-treated DNA, COBRA-Assay and methylation-specific restriction patterns.

In an embodiment, the method according to the present invention further comprises comparing the methylation pattern of the patient's SNCA gene, of intron 1 of the patient's SNCA gene, and/or of the SNCA_{(-926,-483)} region of the patient's SNCA gene, more specifically the methylation status of one or more CpG dinucleotides of the SNCA_{(-926,-483)} region of the patient's SNCA gene, for example, of one or more CpG dinucleotides of the SNCA_{(-926/-483)} region of the patient's SNCA gene selected from the group consisting of CpG dinucleotides No. 1, 2, 7, 8, 11, 12, 13, 18, 21, 22 and 23 of the SNCA_{(-926,-483)} region of the patient's SNCA gene, and, for example, of one or more CpG dinucleotides selected from the group consisting of CpG dinucleotides No. 1, 7, 8, 13, 22 and 23 of the SNCA_{(-926,-483)} region of the patient's SNCA gene, with the methylation status of the corresponding SNCA gene, intron, region and/or CpG dinucleotide(s) of healthy individuals.

In an embodiment, the method according to the present invention further comprises comparing the methylation pattern of the patient's SNCA gene, of intron 1 of the patient's SNCA gene, and/or of the SNCA_{(-926,-483)} region of the patient's SNCA gene, more specifically of one or more CpG dinucleotides of the SNCA_{(-926,-483)} region of the patient's SNCA gene, for example, the methylation status of one or more CpG dinucleotides of the SNCA_{(-926/-483)} region of the patient's SNCA gene selected from the group consisting of CpG dinucleotides No. 1, 2, 7, 8, 11, 12, 13, 18, 21, 22 and 23 of the SNCA_{(-926,-483)} region of the patient's SNCA gene, and, for example, of one or more CpG dinucleotides selected from the group consisting of CpG dinucleotides No. 1, 7, 8, 13, 22 and 23 of the SNCA_{(-926,-483)} region of the patient's SNCA gene, from DNA of a blood sample obtained from the patient at a given point of time with the methylation status of the corresponding gene, intron, region and/or CpG dinucleotide(s) from DNA of one or more further blood samples of the same patient, wherein said one or more further blood sample(s) was/were obtained at different points of time than said blood sample obtained from the patient at said given point of time. This embodiment permits to monitor alterations in the methylation pattern of the patient's SNCA gene, and/or of the SNCA_{(-926,-483)} region of the patient's SNCA gene, more specifically of one or more CpG dinucleotides of the SNCA_{(-926,-483)} region of the patient's SNCA gene, for example, of one or more CpG dinucleotides of the SNCA_{(-926,-483)} region of the patient's SNCA gene selected from the group consisting of CpG dinucleotides No. 1, 2, 7, 8, 11, 12, 13, 18, 21, 22 and 23 of the SNCA_{(-926,-483)} region of the patient's SNCA gene, and, for example, of one or more CpG dinucleotides selected from the group consisting of CpG dinucleotides No. 1, 7, 8, 13, 22 and 23 of the SNCA_{(-926,-483)} region of the patient's SNCA gene, over time, and may enable to elucidating the etiology of Parkinson's disease and/or monitoring progression or regression of Parkinson's disease of a patient.

The method according to the present invention further comprises determining whether the patient's SNCA gene, intron 1 of the patient's SNCA gene, or the SNCA_{(-926,-483)} region of the patient's SNCA gene is hypomethylated compared to the corresponding gene, intron or region of the SNCA gene from healthy individuals. Hypomethylation of the patient's SNCA gene, of intron 1 of the patient's SNCA gene, or of the SNCA_{(-926,-483)} region of the patient's SNCA gene may be determined in that the methylation status of one or more CpG dinucleotides of the SNCA_{(-926,-483)} region of the patient's SNCA gene is compared to the methylation status of the corresponding CpG dinucleotide(s) of the SNCA_{(-926,-483)} region from healthy individuals. In an embodiment, hypomethylation can, for example, be determined in that the methylation status of one or more CpG dinucleotides of the SNCA_{(-926,-483)} region of the patient's SNCA gene selected from the group consisting of CpG dinucleotides No. 1, 7, 8, 13, 22 and 23 of the SNCA_{(-926,-483)} region of the patient's SNCA gene is compared to the methylation status of the corresponding CpG dinucleotide(s) of the SNCA_{(-926,-483)} region from healthy individuals.

In determining whether the patient's SNCA gene, intron 1 of the patient's SNCA gene or the SNCA_{(-926,-483)} region of the patient's SNCA gene is hypomethylated compared to the corresponding gene, intron or region of healthy individuals it can be deduced whether the patient is at risk of developing Parkinson's disease even if neurological symptoms are absent, or whether the patient already has Parkinson's disease even no neurological symptoms are present or only present mildly and yet non-specifically.

In an embodiment of the present invention, the method comprises determining whether the patient's SNCA gene, intron 1 of the patient's SNCA gene, or the SNCA_{(-926,-483)} region of the patient's SNCA gene is hypermethylated with respect to particular CpG dinucleotides within said SNCA gene, said intron 1 or said SNCA_{(-926,-483)} region compared to the corresponding gene, intron or region of the SNCA gene from healthy individuals. Hypermethylation of said SNCA gene, said intron 1 or said SNCA_{(-926,-483)} region with respect to particular CpG dinucleotides therein may be determined in that the methylation status of one or more CpG dinucleotides of the SNCA_{(-926,-483)} region of the patient's SNCA gene is compared to the methylation status of the corresponding CpG dinucleotide(s) of the SNCA_{(-926/-483)} region from healthy individuals. In an embodiment, hypermethylation can, for example, be determined in that the methylation status of one or more CpG dinucleotides selected from the group consisting of CpG dinucleotides No. 2, 11, 12, 18 and 21 of the SNCA_{(-926,-483)} region of the patient's SNCA gene is/are compared to the methylation status of the corresponding CpG dinucleotide(s) of the SNCA_{(-926,-483)} region from healthy individuals.

In determining whether the patient's SNCA gene, intron 1 of the patient's SNCA gene or the SNCA_{(-926,-483)} region of the patient's SNCA gene is hypermethylated with respect to particular CpG dinucleotides within the SNCA gene, intron 1 of the SNCA gene or the SNCA_{(-926/-483)} region compared to the corresponding gene, intron or region of healthy individuals it can be deduced whether the patient is at risk of developing Parkinson's disease even if neurological symptoms are absent, or whether the patient already has Parkinson's disease even no neurological symptoms are present or only present mildly and yet non-specifically.

The present invention will now be described with respect to particular embodiments and with reference to the Figures, however, the present invention is not limited thereto. The drawings described are only schematic and are non-limiting.

FIG. 1 displays the nucleotide sequence of SNCA_{(-926,-483)} (SEQ ID No. 1) which is a fragment of intron I of the human SNCA gene, ranging from nucleotide position −926 to nucleotide position −483 upstream of the SNCA start codon (nucleotide positions 1 to 3). Within SNCA_{(-926,-483)} 23 CpG dinucleotides are present which may be subject to methylation. Said 23 CpG dinucleotides are identified in the nucleotide sequence in that they are displayed in bold letters. The 23 CpG dinucleotides are consecutively enumerated, from distant to proximal with respect to the SNCA start codon. Predicted transcription factor binding sites within the SNCA_{(-926,-483)} adjacent to CpG dinucleotides are designated below the nucleotide sequence.

FIG. 2 shows a diagram illustrating the methylation pattern of the 23 CpG dinucleotides within the SNCA_{(-926,-483)} region from peripheral blood monocytes DNA. White columns display the percentage of methylation of the cytosine of the CpG dinucleotide indicated on the X-axis from healthy individuals whereas black columns display the percentage of methylation of the cytosine of the CpG dinucleotide indicated on the X-axis from patients affected with sporadic PD. Mean values are indicated by the height of each column and standard deviations are indicated. Asterisks indicate statistical significance of the obtained results with *p<0.05 and **p<0.01.

### EXAMPLES

Peripheral blood monocytes were isolated from blood samples of 19 patients suffering from sporadic PD and 20 healthy individuals. DNA isolation and bisulfite treatment of the isolated DNA were performed as described by Pieper et al. (Pieper, H. C. et al. (2008), Different methylation of the TNF-alpha promoter in cortex and substantia nigra: implications for selective neuronal vulnerability, Neurobiol. Dis. 32:521-527).

Bisulfite treated DNA was amplified using SNCA—For (SEQ ID NO: 2) and SNCA-Rev (SEQ ID NO: 3), specifically designed for bisulfite treated SNCA_{(-926/-483)} DNA. PCR products were cloned into pCR 2.1 TOPO vector (Invitrogen) following the manufacturer's instructions. Plasmid DNA was isolated from at least ten clones per individual, and sequenced using vector specific primer M13reverse (CAGGAAACAGCTATGAC; SEQ ID NO: 4) and the Big Dye Terminator v1.1 Cycle Sequencing Kit (Applied Biosystems) and analyzed on an ABI PRISM 310 Genetic Analyzer.

The results of determining the methylation pattern of the SNCA_{(-926/-483)} DNA from lymphocytes of healthy individuals and PD patients is shown in FIG. 2.

FIG. 2 compares the methylation status of the 23 CpG dinucleotides within the SNCA_{(-926,-483)} region of the human SNCA gene from peripheral blood monocytes of patients who developed Parkinson's disease with those of healthy individuals. As can be inferred from FIG. 2, the percentage of methylation of CpG dinucleotide Nos. 1, 7, 8, 13, 22 and 23 is significantly lower in patients affected with PD compared to healthy control individuals. The SNCA gene, in particular intron 1 of the human SNCA genes, more specifically the SNCA_{(-926/-483)} region, is therefore significantly hypomethylated with respect to CpG dinucleotides No. 1, 7, 8, 13 and 23. Non-methylation of one or more dinucleotides selected from the group consisting of CpG dinucleotides No. 1, 7, 8, 13, 22 and 23 of the SNCA_{(-926/-483)} region obtained from DNA of peripheral blood monocytes of a patient may thus indicate that said patient is at higher risk of developing Parkinson's disease or already developed Parkinson's disease even if no symptoms are present at that stage of the disease.

FIG. 2 additionally indicates that the percentage of CpG dinucleotides No. 2, 11, 12, 18 and 21 being methylated is higher in the SNCA_{(-926,-483)} region of the human SNCA gene obtained from peripheral blood monocytes of PC patients than in that obtained from healthy individuals. Although statistically not yet significant, the tendency is striking, and analyzing additional samples will render this observation statistically significant. Methylation of CpG dinucleotides No. 2, 11, 12, 18 and 21 of the SNCA_{(-926,-483)} region obtained from DNA of peripheral blood monocytes of a patient may thus indicate that said patient is at higher risk of developing Parkinson's disease or already developed Parkinson's disease even if no symptoms are present at that stage of the disease.

Overall, methylation of the human SNCA gene from peripheral blood monocytes, in particular of intron 1 of the human SNCA genes, and more specifically of the SNCA_{(-926,-483)} region, is reduced in PD patients compared to control. Linear regression analysis furthermore denoted a trend towards hypomethylation with increased age in PD patients.

While the present invention has been illustrated and described in detail in the drawings and foregoing description, such illustration and description are to be considered illustrative or exemplary and not restrictive; the present invention is not limited to the disclosed embodiments. Other variations to the disclosed embodiments can be understood and effected by those skilled in the art in practicing the present invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

What is claimed is:

1-10. (canceled)

11. A method of using peripheral blood monocytes of a patient to at least one of diagnose and assess a risk of a patient of developing Parkinson's disease, the method comprising:
obtaining peripheral blood monocytes from the patient; and
determining a methylation pattern of the SNCA_{(-926,-483)} region of the SNCA gene of the DNA of the peripheral blood monocytes.

12. The method of using as recited in claim 11, further comprising:
determining a methylation status of at least one CpG dinucleotide of the SNCA_{(-926/-483)} region,
wherein the at least one CpG dinucleotide is selected from a group consisting of CpG dinucleotides No. 1, 2, 7, 8, 11, 12, 13, 18, 21, 22 and 23.

13. The method of using as recited in claim 12, wherein the methylation status is determined by at least one method selected from a group consisting of:
methylation specific PCR,
a sequence analysis of bisulfite-treated DNA,
a COBRA-Assay, and
methylation-specific restriction patterns.

14. A method of determining a methylation pattern of the SNCA_{(-926,-483)} region of the human SNCA gene to at least one of diagnose and assess a risk of a patient of developing Parkinson's disease, the method comprising:
obtaining peripheral blood monocytes from the patient; and
obtaining the SNCA gene from DNA of the peripheral blood monocytes.

15. The method as recited in claim 14, further comprising:
determining a methylation status of at least one CpG dinucleotide of a SNCA_{(-926,-483)} region, wherein the at least one CpG dinucleotide is selected from a group consisting of CpG dinucleotides No. 1, 2, 7, 8, 11, 12, 13, 18, 21, 22 and 23.

16. The method as recited in claim 15, wherein the methylation status is determined by at least one method selected from a group consisting of:
methylation specific PCR,
a sequence analysis of bisulfite-treated DNA,
a COBRA-Assay, and
methylation-specific restriction patterns.
